(19) United States
(12) Patent Application Publication
(10) Pub. No.: US 2005/0065154 A1
(43) Pub. Date: Mar. 24, 2005

(54) Treatment of migraine accompanied by nausea with a combination of cyclooxygenase-2 selective inhibitors and anti-nausea agents

(75) Inventor: Karen Siebert, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/875,940

(22) Filed: Jun. 24, 2004

Related U.S. Application Data
(60) Provisional application No. 60/480,939, filed on Jun. 24, 2003.

(51) Int. Cl. A61K 31/60; A61K 31/50; A61K 31/425; A61K 31/415

(52) U.S. Cl. 514/247; 514/406; 514/372

(57) ABSTRACT

The present invention is related to the treatment or prevention of migraine accompanied by nausea or vomiting with a combination of a cyclooxygenase-2 selective inhibitor and an anti-nausea agent.

### CROSS REFERENCE TO RELATED APPLICATION

This application claims priority from Provisional Application Ser. No. 60/480,939 filed on Jun. 24, 2003, which is hereby incorporated by reference in its entirety.

### FIELD OF INVENTION

The present invention relates to the treatment of a migraine accompanied by nausea or vomiting. In particular, the invention is directed toward a combination therapy comprising the administration to a subject of a COX-2 selective inhibitor and an anti-nausea agent.

### BACKGROUND OF THE INVENTION

An estimated 23 to 25 million Americans—about 18% of women and 6% of men—suffer from migraine pain and migraine-related symptoms. Attacks are common, with more than 50% of sufferers experiencing one or more episodes per month.

Migraine, a heterogeneous disorder, produces a wide spectrum of pain and associated disabilities, both within and among individual sufferers. The spectrum includes mild pain and no disability in approximately 5-15% of migraine attacks, moderate to severe pain and disability in approximately 60-70% of attacks, and incapacitating pain and total disability in the remaining approximately 25-35% of attacks.

Many migraine sufferers use single-agent nonprescription analgesics such as acetaminophen, or aspirin to treat their attacks. Despite the widespread use of nonprescription drugs for self-treatment, only prescription drugs are approved for the treatment of migraine in the United States. In other countries, a number of nonprescription drugs are specifically approved for migraine pain; however, the effectiveness of self-treatment of migraine and the effectiveness of such nonprescription drugs in relieving or aborting migraine pain and/or the characteristic symptoms of migraine has not been adequately studied in well-controlled clinical trials. The combination of acetaminophen, aspirin and caffeine is approved for relief of nonspecific headaches and tension headaches, which are clinical and physiologically distinct from migraine. Caffeine is an analgesic adjuvant for a variety of pain conditions and has been included in combination with other analgesics, ergot alkaloids and barbiturates in prescription formulations for migraine.

Non-steroidal antiinflammatory drugs (NSAIDs) are active in reducing the prostaglandin-induced pain and swelling associated with the inflammation process but are also active in affecting other prostaglandin-regulated processes not associated with the inflammation process. Thus, use of high doses of most common NSAIDs can produce severe side effects, including life-threatening ulcers that limit their therapeutic potential. An alternative to NSAIDs is the use of corticosteroids, which have even more drastic side effects, especially when long-term therapy is involved.

Previous NSAIDs have been found to prevent the production of prostaglandin by inhibiting enzymes in the human arachidonic acid/prostaglandin pathway including the enzyme cyclooxygenase (COX). The more recent discovery that there are two isoforms of the COX enzyme, the first, (COX-1), being involved with physiological functions and the second, COX-2, being induced in inflamed tissue, has given rise to a new approach. The severe side effects sometimes associated with treatment with NSAIDs, particularly ulcerations and bleeding in the gastrointestinal tract, are ascribed to the inhibition of COX-1. While conventional NSAIDs block both forms of the enzyme, the identification of the inducible COX-2 enzyme associated with inflammation has provided a viable target of inhibition that more effectively reduces inflammation, relieves pain, and produces fewer and less drastic side effects.

Compounds that selectively inhibit cyclooxygenase-2 have been described in U.S. Pat. Nos. 5,380,738; 5,344,991; 5,393,790; 5,434,178; 5,474,995; 5,510,368 and PCT publications WO96/06840, WO96/03388, WO96/03387, WO96/19469, WO96/25405, WO95/15316, WO94/15932, WO94/27980, WO95/00501, WO94/13635, WO94/20480, and WO94/26731. [Pyrazol-1-yl]benzenesulfonamides have been described as inhibitors of cyclooxygenase-2 and have shown promise in the treatment of inflammation, arthritis, and pain, with minimal side effects in pre-clinical and clinical trials. Their use for treating inflammation in vascular disease has been described in U.S. Pat. No. 5,466,823. Their use for preventing cardiovascular-related diseases has been described in co-pending U.S. patent application Ser. No. 09/402,634.

Although the symptom pattern varies among migraine sufferers, the severity of migraine pain justifies a need for vigorous therapy in the great majority of cases. Nausea symptoms commonly accompany migraine attacks in many individuals. Combined preparations of paracetamol or aspirin with an anti-nausea agent such as buclizine or metoclopramide, have been used to alleviate the nausea symptoms that often accompanied a migraine attack. Commercially, they are available as Migraleve Duo, Paramax, and Migravess. Narcotic analgesics such as codeine have also been employed together with NSAIDs to obtain synergistic analgesia, for example Migraleve Yellow, and co-codamol. Also, a combination of NSAIDs and domperidone or an analogue thereof to combat nausea associated with migraine attack has been used. See U.S. Pat. No. 6,319,514.

### SUMMARY OF THE INVENTION

Among the several aspects of the invention is provided a method and a composition for the treatment, prevention, or inhibition of migraine headaches and associated nausea in a subject. The composition comprises a cyclooxygenase-2 selective inhibitor or an isomer, a pharmaceutically acceptable salt, ester, or a prodrug thereof and an anti-nausea agent, and the method comprises administering to the subject a cyclooxygenase-2 selective inhibitor or an isomer, ester, a pharmaceutically acceptable salt or a prodrug thereof in combination with an anti-nausea agent.

In one embodiment, the cyclooxygenase-2 selective inhibitor is a member of the chromene class of compounds. For example, the chromene compound may be a compound or an isomer, ester, a pharmaceutically acceptable salt or a prodrug thereof of the formula: 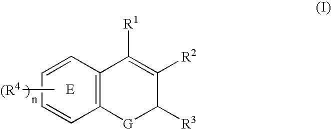
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl, or wherein R⁴ together with the carbon atoms to which it is attached and the remainder of ring E forms a naphthyl radical.

In another embodiment, the cyclooxygenase-2 selective inhibitor or an isomer, ester, a pharmaceutically acceptable salt or a prodrug thereof comprises a compound of the formula: 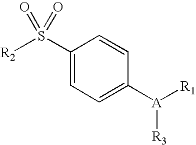
wherein
A is selected from the group consisting of a partially unsaturated or unsaturated heterocyclyl ring and a partially unsaturated or unsaturated carbocyclic ring;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and

R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

In another embodiment, the anti-nausea agent is selected from the group consisting of anticholinergics (i.e., scopolamine), antihistamines (i.e., dimenhydrinate, diphenhydramine, hydroxyzine), benzodiazepines (i.e., diazepam, lorazepam), phenothiazines (i.e., chlorpromazine, methotrimeprazine, perphenazine, prochlorperazine, promethazine, trifluoperazine, triflupromazine), benzquinamide, bismuth subsalicylate, buclizine, cinnarizine, cyclizine, diphenidol, dolasetron, domperidone, dronabinol, droperidol, granisetron, haloperidol, meclizine, metoclopramide, nabilone, ondansetron, thiethylperazine, trimethobenzamide, and eziopitant.

In a further embodiment, the cyclooxygenase-2 selective inhibitor or an isomer, a pharmaceutically acceptable salt, ester, or prodrug thereof is administered during a continuous period beginning prior to the administration of the anti-nausea agent.

Other objects and features will be in part apparent and in part pointed out hereinafter.

Abbreviations and Definitions

Generally, the nomenclature used hereafter, and the laboratory procedures are those well known and commonly employed in the art. Unless defined otherwise, all technical and scientific terms used herein have the same meaning as commonly understood by one of ordinary skill in the art to which this invention relates. Although any methods and materials similar or equivalent to those described herein can be used in the practice or testing of the present invention, the preferred methods and materials are described. To facilitate understanding of the invention, a number of terms as used herein are defined below:

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The phrase “administration” in defining the use of both a cyclooxygenase-2 inhibitor and anti-nausea agent is intended to embrace administration of each agent in a manner and in a regimen that will provide beneficial effects of the drug combination therapy, and is intended as well to embrace co-administration of 2 or more of the COX-2 agents in a substantially simultaneous manner and/or 2 or more of the anti-nausea agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from the constituent COX-2 agent and anti-nausea agent used in combination.

The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxycarbonyl” is a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkylamino” is an amino group that has been substituted with one or two alkyl radicals. Preferred is “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred is “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” is a radical containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radicals. More preferred are “lower aminoalkyl” radicals. Examples of such radicals include aminomethyl, aminoethyl, and the like.

The term “aminocarbonyl” is an amide group of the formula —C(═O)NH2.

The term “aralkoxy” is an aralkyl radical attached through an oxygen atom to other radicals.

As used herein, the term “anti-nausea agent” refers to an agent that has an anti-nausea or anti-emetic effect. “Anti-nausea agent” thus means an agent that (1) prevents nausea or vomiting from occurring in a subject who may be predisposed to the nausea or vomiting; (2) inhibits nausea or vomiting in a subject who may be experiencing nausea or vomiting; or (3) ameliorates or relieves the symptoms of the nausea or vomiting. Some anti-nausea agents include, but are not limited to, dimenhydrinate (dramamine), domperidone, scopolamine (hyoscine), cinnarizine, metoclopramide, cyclizine, and promethazine.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “aralkylamino” is an aralkyl radical attached through an amino nitrogen atom to other radicals. The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aralkylthio” is an aralkyl radical attached to a sulfur atom.

The term “aralkylthioalkyl” is an aralkylthio radical attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl”, alone or in combination, is a carbocyclic aromatic system containing one, two or three rings wherein such rings may be attached together in a pendent manner or may be fused. The term “aryl” includes aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “arylamino” is an amino group, which has been substituted with one or two aryl radicals, such as N-phenylamino. The “arylamino” radicals may be further substituted on the aryl ring portion of the radical.

The term “aryloxyalkyl” is a radical having an aryl radical attached to an alkyl radical through a divalent oxygen atom.

The term “arylthioalkyl” is a radical having an aryl radical attached to an alkyl radical through a divalent sulfur atom.

The term “carbonyl”, whether used alone or with other terms, such as “alkoxycarbonyl”, is —(C═O)—.

The terms “carboxy” or “carboxyl”, whether used alone or with other terms, such as “carboxyalkyl”, is —CO2H.

The term “carboxyalkyl” is an alkyl radical substituted with a carboxy radical. More preferred are “lower carboxyalkyl” which are lower alkyl radicals as defined above, and may be additionally substituted on the alkyl radical with halo. Examples of such lower carboxyalkyl radicals include carboxymethyl, carboxyethyl and carboxypropyl.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “cyclooxygenase-2 selective inhibitor” is a compound able to inhibit cyclooxygenase-2 without significant inhibition of cyclooxygenase-1. Typically, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more typically, of at least 100. Even more typically, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “halo” is a halogen such as fluorine, chlorine, bromine or iodine.

The term “haloalkyl” is a radical wherein any one or more of the alkyl carbon atoms is substituted with halo as defined above. Specifically included are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” is a radical having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also includes radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “hydrido” is a single hydrogen atom (H). This hydrido radical may be attached, for example, to an oxygen atom to form a hydroxyl radical or two hydrido radicals may be attached to a carbon atom to form a methylene (—CH2-) radical.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prevention” includes preventing a clinically evident migraine with associated nausea altogether. This definition includes prophylactic treatment. Such prophylactic treatment includes recognizing the early stages of migraine, and using the compositions or practicing the methods of the present invention so as to prevent the occurrence of said migraine with associated nausea.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The term “subject” for purposes of treatment includes any human or animal who is susceptible to a migrane with associated nausea. The subject can be a domestic livestock species, a laboratory animal species, a zoo animal or a companion animal. In one embodiment, the subject is a mammal. In one embodiment, the mammal is a human being.

The term “sulfonyl”, whether used alone or linked to other terms such as alkylsulfonyl, is a divalent radical —SO₂—. “Alkylsulfonyl” is an alkyl radical attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrases “therapeutically-effective” and “effective for the treatment, prevention, or inhibition,” are intended to qualify the amount of each agent (i.e. the amount of cyclooxygenase-2 selective inhibitor and the amount of anti-nausea agent) that will achieve the goal of improvement in disorder severity and the frequency of incidence over no treatment or treatment of each agent by itself.

The term “treat” or “treatment” as used herein, includes administration of the combination therapy to a subject known to have migraine headaches. In other aspects, it also includes either preventing the onset of a clinically evident migraine or preventing the onset of a preclinically evident stage of a migraine. This definition includes prophylactic treatment.

### DESCRIPTION OF THE PREFERRED EMBODIMENTS

The present invention provides a combination therapy comprising the administration to a subject of a therapeutically effective amount of a COX-2 selective inhibitor in combination with a therapeutically effective amount of an anti-nausea agent. The combination therapy is used to treat or prevent a migraine accompanied by nausea and/or vomiting. When administered as part of a combination therapy, the COX-2 selective inhibitor together with the anti-nausea agent provides enhanced treatment options as compared to administration of either the anti-nausea agent alone or the COX-2 selective inhibitor alone.

Cyclooxygenase-2 Selective Inhibitors

A number of suitable cyclooxygenase-2 selective inhibitors or an isomer, a pharmaceutically acceptable salt, ester, or prodrug thereof, may be employed in the composition of the current invention. In one embodiment, the cyclooxygenase-2 selective inhibitor can be, for example, the cyclooxygenase-2 selective inhibitor meloxicam, Formula B-1 (CAS registry number 71125-38-7) or an isomer, a pharmaceutically acceptable salt, ester, or prodrug of a compound having Formula B-1.

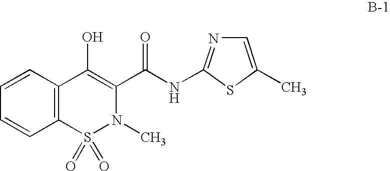

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or an isomer, a pharmaceutically acceptable salt, ester, or prodrug of a compound having Formula B-2.

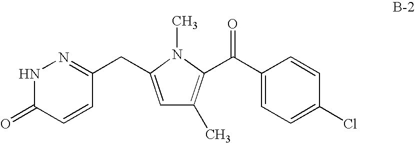

In still another embodiment the cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having

Formula I shown below and possessing, by way of example and not limitation, the structures disclosed in Table 1. Furthermore, benzopyran cyclooxygenase-2 selective inhibitors useful in the practice of the present methods are described in U.S. Pat. Nos. 6,034,256 and 6,077,850 herein incorporated by reference in their entirety.

In another embodiment, the cyclooxygenase-2 selective inhibitor is a chromene compound represented by Formula I or an isomer, a pharmaceutically acceptable salt, ester, or prodrug thereof: 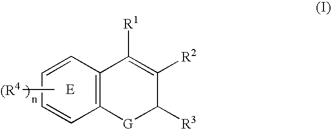
wherein:
n is an integer which is 0, 1, 2, 3 or 4;
G is O, S or NR^{a};
R^{a} is alkyl;

R¹ is selected from the group consisting of H and aryl;
R² is selected from the group consisting of carboxyl, lower alkyl, lower aralkyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from the group consisting of alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,
wherein:
n is an integer which is 0, 1, 2, 3 or 4;
G is O, S or NR^{a};
R^{a} is alkyl;
R¹ is H;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with the carbon atoms to which it is attached and the remainder of ring E forms a naphthyl radical.

In a further embodiment, the cyclooxygenase-2 selective inhibitor may also be a compound of Formula (I), or an isomer, a pharmaceutically acceptable salt, ester, or prodrug thereof,
wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is independently H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,
wherein:
n is an integer which is 0, 1, 2, 3 or 4;
G is oxygen or sulfur;
R¹ is H;
R² is carboxyl;
R³ is lower haloalkyl; and
each R⁴ is independently H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,
wherein:
n is an integer which is 0, 1, 2, 3 or 4;
G is oxygen or sulfur;
R¹ is H;
R² is carboxyl;
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is independently H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,
wherein:
n is an integer which is 0, 1, 2, 3 or 4;
G is oxygen or sulfur;
R¹ is H;
R² is carboxyl;
R³ is trifluoromethyl or pentafluoroethyl; and
each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof,
wherein:
n is 4;
G is O or S;
R¹ is H;
R² is CO₂H;
R³ is lower haloalkyl;
a first R⁴ corresponding to R⁹ is hydrido or halo;
a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;
a third R⁴ corresponding to R¹¹ is H, lower alkyl, halo, lower alkoxy, or aryl; and
a fourth R⁴ corresponding to R¹² is H, halo, lower alkyl, lower alkoxy, or aryl;
wherein Formula (I) is represented by Formula (Ia):

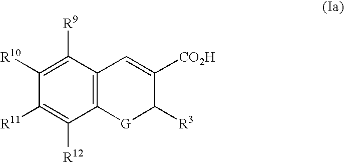

The cyclooxygenase-2 selective inhibitor used in connection with the method(s) of the present invention can also be a compound of having the structure of Formula (Ia) or an isomer, a pharmaceutically acceptable salt, ester, or prodrug thereof,
wherein:
G is O or S;
R³ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

Examples of exemplary chromene cyclooxygenase-2 selective inhibitors are depicted in Table 1 below.

TABLE 1
EXAMPLES OF CHROMENE CYCLOOXYGENASE-2 SELECTIVE
INHIBITORS AS EMBODIMENTS

| Compound | |
|---|---|
| Number | Structural Formula |
| B-3 | 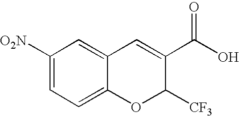 6-Nitro-2-trifluoromethyl-2H-1- benzopyran-3-carboxylic acid |
| B-4 | 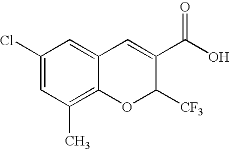 6-Chloro-8-methyl-2-trifluoromethyl- 2H-1-benzopyran-3-carboxylic acid |
| B-5 | 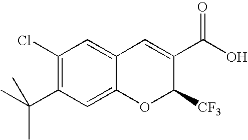 ((S)-6-Chloro-7-(1,1-dimethylethyl)-2-(trifluo- romethyl-2H-1-benzopyran-3-carboxylic acid |
| B-6 | 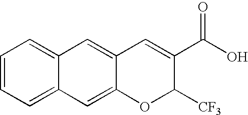 2-Trifluoromethyl-2H-naphtho[2,3-b]pyran-3-carboxylic acid |
| B-7 | 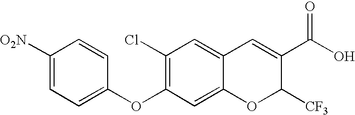 6-Chloro-7-(4-nitrophenoxy)-2-(trifluoromethyl)-2H-1- benzopyran-3-carboxylic acid |
| B-8 | 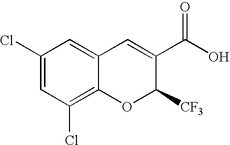 ((S)-6,8-Dichloro-2-(trifluoromethyl)- 2H-1-benzopyran-3-carboxylic acid |
| B-9 | 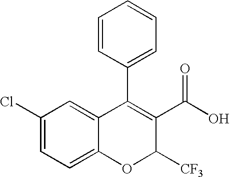 6-Chloro-2-(trifluoromethyl)-4-phenyl-2H- 1-benzopyran-3-carboxylic acid |
| B-10 | 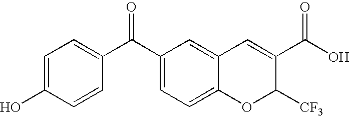 6-(4-Hydroxybenzoyl)-2-(trifluoromethyl)- 2H-1-benzopyran-3-carboxylic acid |
| B-11 | 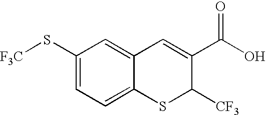 2-(Trifluoromethyl)-6-[(trifluoromethyl)thiol]- 2H-1-benzothiopyran-3-carboxylic acid |
| B-12 | 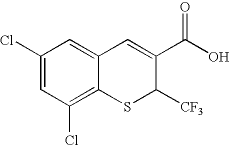 6,8-Dichloro-2-trifluoromethyl-2H-1- benzothiopyran-3-carboxylic acid |
| B-13 | 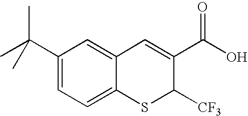 6-(1,1-Dimethylethyl)-2-(trifluoromethyl)- 2H-1-benzothiopyran-3-carboxylic acid |
| B-14 | 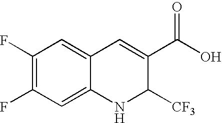 6,7-Difluoro-1,2-dihydro-2-(trifluoro methyl)-3-quinolinecarboxylic acid |
| B-15 | 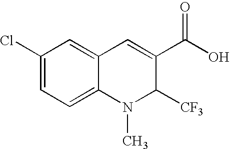 6-Chloro-1,2-dihydro-1-methyl-2-(trifluoro methyl)-3-quinolinecarboxylic acid |
| B-16 | 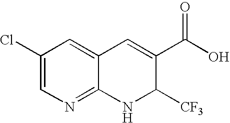 6-Chloro-2-(trifluoromethyl)-1,2-dihydro [1,8]naphthyridine-3-carboxylic acid |
| B-17 | 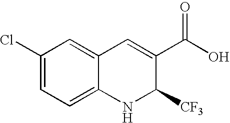 ((S)-6-Chloro-1,2-dihydro-2-(trifluoro methyl)-3-quinolinecarboxylic acid |

In a further embodiment, the cyclooxygenase-2 selective inhibitor is selected from the class of tricyclic cyclooxygenase-2 selective inhibitors represented by the general structure of Formula II or an isomer, a pharmaceutically acceptable salt, ester, or prodrug thereof, 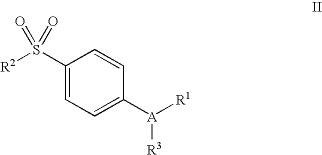
wherein:
A is selected from the group consisting of a partially unsaturated or unsaturated heterocyclyl ring and a partially unsaturated or unsaturated carbocyclic ring;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

In another embodiment, the cyclooxygenase-2 selective inhibitor represented by the above Formula II is selected from the group of compounds illustrated in Table 2, consisting of celecoxib (B-18; U.S. Pat. No. 5,466,823; CAS No. 16959042-5), valdecoxib (B-1 g; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), tilmacoxib (JTE-522; B-23; CAS No. 180200-684).

TABLE 2
EXAMPLES OF TRICYCLIC CYCLOOXYGENASE-2 SELECTIVE IN-
HIBITORS AS EMBODIMENTS

| Compound | |
|---|---|
| Number | Structural Formula |
| B-18 | 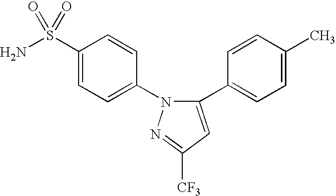 |
| B-19 | 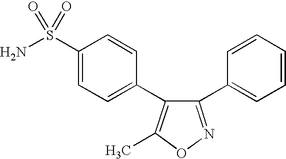 |
| B-20 | 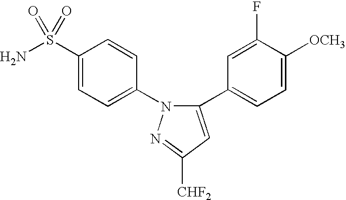 |
| B-21 | 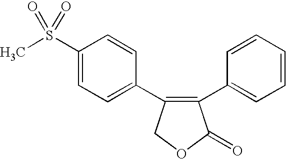 |
| B-22 | 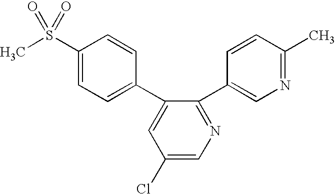 |
| B-23 | 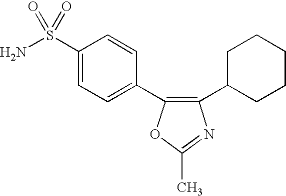 |

In still another embodiment, the cyclooxygenase-2 selective inhibitor is selected from the group consisting of celecoxib, rofecoxib and etoricoxib.

In yet another embodiment, the cyclooxygenase-2 selective inhibitor is parecoxib (B-24, U.S. Pat. No. 5,932,598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

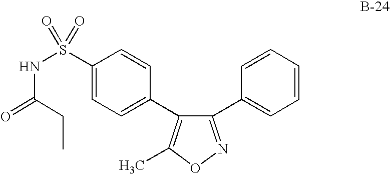

One form of parecoxib is sodium parecoxib.

In another embodiment of the invention, the compound having the formula B-25 or an isomer, a pharmaceutically acceptable salt, ester, or prodrug of a compound having formula B-25 that has been previously described in International Publication number WO 00/24719 (which is herein incorporated by reference) is another tricyclic cyclooxygenase-2 selective inhibitor that may be advantageously employed.

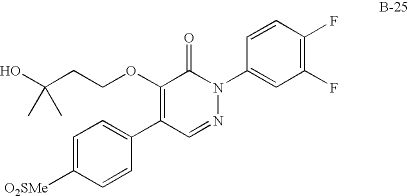

Another cyclooxygenase-2 selective inhibitor that is useful in connection with the method(s) of the present invention is N-(2-cyclohexyloxynitrophenyl)-methane sulfonamide (NS-398) having a structure shown below as B-26, or an isomer, a pharmaceutically acceptable salt, ester, or prodrug of a compound having formula B-26.

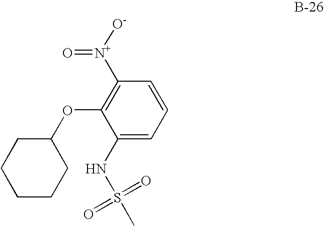

In yet a further embodiment, the cyclooxygenase-2 selective inhibitor used in connection with the method(s) of the present invention can be selected from the class of phenylacetic acid derivative cyclooxygenase-2 selective inhibitors represented by the general structure of Formula (III) or an isomer, a pharmaceutically acceptable salt, ester, or prodrug thereof: 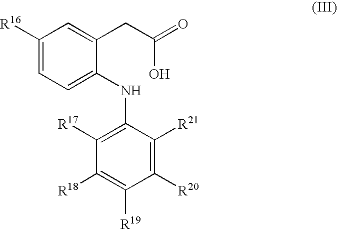
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl, provided, however, that each of R¹⁷, R¹⁸, R²⁰ and R²¹ is not fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (lumiracoxib; B-211) and that has the structure shown in Formula (III) or an isomer, a pharmaceutically acceptable salt, ester, or prodrug thereof wherein:
R¹⁶ is ethyl;
R¹⁷ and R¹⁹ are chloro;
R¹⁸ and R²⁰ are hydrogen; and
R²¹ is methyl.

In yet another embodiment, the cyclooxygenase-2 selective inhibitor is represented by Formula (IV) or an isomer, a pharmaceutically acceptable salt, ester, or prodrug thereof: 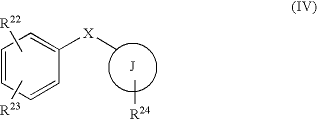
wherein:
X is O or S;
J is a carbocycle or a heterocycle;
R²² is NHSO₂CH₃ or F;
R²³ is H, NO₂, or F; and

R²⁴ is H, NHSO₂CH₃, or (SO₂CH₃)C₆H₄.

According to another embodiment, the cyclooxygenase-2 selective inhibitors used in the present method(s) have the structural Formula (V) or an isomer, a pharmaceutically acceptable salt, ester, or prodrug thereof: 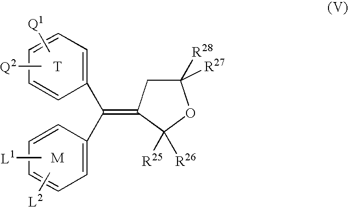
wherein:
T and M are independently phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or
R²⁵ and R²⁶, together with the carbon atom to which they are attached, form a carbonyl or a saturated hydrocarbon ring having from 3 to 7 carbon atoms; or
R²⁷ and R²⁸, together with the carbon atom to which they are attached, form a carbonyl or a saturated hydrocarbon ring having from 3 to 7 carbon atoms;
Q¹; Q², L¹ or L² are independently hydrogen, halogen, lower alkyl having from 1 to 6 carbon atoms, trifluoromethyl, lower methoxy having from 1 to 6 carbon atoms, alkylsulfinyl or alkylsulfonyl; and
at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or
Q¹ and Q² together form methylenedioxy; or
L¹ and L² together form methylenedioxy.

In another embodiment, the compounds N-(2-cyclohexyloxynitrophenyl) methane sulfonamide, and (E)-4-[(4-methylphenyl)(tetrahydro-2-oxo-3-furanylidene) methyl]benzenesulfonamide or an isomer, a pharmaceutically acceptable salt, ester, or prodrug thereof having the structure of Formula (V) are employed as cyclooxygenase-2 selective inhibitors.

In a further embodiment, compounds that are useful for the cyclooxygenase-2 selective inhibitor or an isomer, a pharmaceutically acceptable salt, ester, or prodrug thereof used in connection with the method(s) of the present invention, the structures for which are set forth in Table 3 below, include, but are not limited to:
6-chloro-2-trifluoromethyl-2H-1-benzopyran-3-carboxylic acid (B-27);
6-chloro-7-methyl-2-trifluoromethyl-2H-1-benzopyran-3-carboxylic acid (B-28);
8-(1-methylethyl)-2-trifluoromethyl-2H-1-benzopyran-3-carboxylic acid (B-29);
6-chloro-8-(1-methylethyl)-2-trifluoromethyl-2H-1-benzopyran-3-carboxylic acid (B-30);
2-trifluoromethyl-3H-naphtho[2,1-b]pyran-3-carboxylic acid (B-31);
7-(1,1-dimethylethyl)-2-trifluoromethyl-2H-1-benzopyran-3-carboxylic acid (B-32);
6-bromo-2-trifluoromethyl-2H-1-benzopyran-3-carboxylic acid (B-33);
8-chloro-2-trifluoromethyl-2H-1-benzopyran-3-carboxylic acid (B-34);
6-trifluoromethoxy-2-trifluoromethyl-2H-1-benzopyran-3-carboxylic acid (B-35);
5,7-dichloro-2-trifluoromethyl-2H-1-benzopyran-3-carboxylic acid (B-36);
8-phenyl-2-trifluoromethyl-2H-1-benzopyran-3-carboxylic acid (B-37);
7,8-dimethyl-2-trifluoromethyl-2H-1-benzopyran-3-carboxylic acid (B-38);
6,8-bis(dimethylethyl)-2-trifluoromethyl-2H-1-benzopyran-3-carboxylic acid (B-39);
7-(1-methylethyl)-2-trifluoromethyl-2H-1-benzopyran-3-carboxylic acid (B40);
7-phenyl-2-trifluoromethyl-2H-1-benzopyran-3-carboxylic acid (B-41);
6-chloro-7-ethyl-2-trifluoromethyl-2H-1-benzopyran-3-carboxylic acid (B-42);
6-chloro-8-ethyl-2-trifluoromethyl-2H-1-benzopyran-3-carboxylic acid (B-43);
6-chloro-7-phenyl-2-trifluoromethyl-2H-1-benzopyran-3-carboxylic acid (B-44);
6,7-dichloro-2-trifluoromethyl-2H-1-benzopyran-3-carboxylic acid (B45);
6,8-dichloro-2-trifluoromethyl-2H-1-benzopyran-3-carboxylic acid (B-46);
6-chloro-8-methyl-2-trifluoromethyl-2H-1-benzopyran-3-carboxylic acid (B-47);
8-chloro-6-methyl-2-trifluoromethyl-2H-1-benzopyran-3-carboxylic acid (B-48)
8-chloro-6-methoxy-2-trifluoromethyl-2H-1-benzopyran-3-carboxylic acid (B-49);
6-bromo-8-chloro-2-trifluoromethyl-2H-1-benzopyran-3-carboxylic acid (B-50);
8-bromo-6-fluoro-2-trifluoromethyl-2H-1-benzopyran-3-carboxylic acid (B-51);
8-bromo-6-methyl-2-trifluoromethyl-2H-1-benzopyran-3-carboxylic acid (B-52);
8-bromo-5-fluoro-2-trifluoromethyl-2H-1-benzopyran-3-carboxylic acid (B-53);
6-chloro-8-fluoro-2-trifluoromethyl-2H-1-benzopyran-3-carboxylic acid (B-54);
6-bromo-8-methoxy-2-trifluoromethyl-2H-1-benzopyran-3-carboxylic acid (B-55);
6-[[(phenylmethyl)amino]sulfonyl]-2-trifluoromethyl-2H-1-benzopyran-3-carboxylic acid (B-56);
6-[(dimethylamino)sulfonyl]-2-trifluoromethyl-2H-1-benzopyran-3-carboxylic acid (B-57);
6-[(methylamino)sulfonyl]-2-trifluoromethyl-2H-1-benzopyran-3-carboxylic acid (B-58);
6-[(4-morpholino)sulfonyl]-2-trifluoromethyl-2H-1-benzopyran-3-carboxylic acid (B-59);
6-[(1,1-dimethylethyl)aminosulfonyl]-2-trifluoromethyl-2H-1-benzopyran-3-carboxylic acid (B-60);
6-[(2-methylpropyl)aminosulfonyl]-2-trifluoromethyl-2H-1-benzopyran-3-carboxylic acid (B-61);
6-methylsulfonyl-2-trifluoromethyl-2H-1-benzopyran-3-carboxylic acid (B-62);
8-chloro-6-[[(phenylmethyl)amino]sulfonyl]-2-trifluoromethyl-2H-1-benzopyran-3-carboxylic acid (B-63);
6-phenylacetyl-2-trifluoromethyl-2H-1-benzopyran-3-carboxylic acid (B-64);
6,8-dibromo-2-trifluoromethyl-2H-1-benzopyran-3-carboxylic acid (B-65);
8-chloro-5,6-dimethyl-2-trifluoromethyl-2H-1-benzopyran-3-carboxylic acid (B-66);
6,8-dichloro-(S)-2-trifluoromethyl-2H-1-benzopyran-3-carboxylic acid (B-67);
6-benzylsulfonyl-2-trifluoromethyl-2H-1-benzopyran-3-carboxylic acid (B-68);
6-[[N-(2-furylmethyl)amino]sulfonyl]-2-trifluoromethyl-2H-1-benzopyran-3-carboxylic acid (B-69);
6-[[N-(2-phenylethyl)amino]sulfonyl]-2-trifluoromethyl-2H-1-benzopyran-3-carboxylic acid (B-70);
6-iodo-2-trifluoromethyl-2H-1-benzopyran-3-carboxylic acid (B-71);
7-(1,1-dimethylethyl)-2-pentafluoroethyl-2H-1-benzopyran-3-carboxylic acid (B-72);
6-chloro-2-trifluoromethyl-2H-1-benzothiopyran-3-carboxylic acid (B-73);
3-[(3-chloro-phenyl)-(4-methanesulfonyl-phenyl)-methylene]-dihydro-furan-2-one or BMS-347070 (B-74);
8-acetyl-3-(4-fluorophenyl)-2-(4-methylsulfonyl)phenyl-imidazo(1,2-a)pyridine (B-75);
5,5-dimethyl-4-(4-methylsulfonyl)phenyl-3-phenyl-2-(5H)-furanone (B-76);
5-(4-fluorophenyl)-1-[4-(methylsulfonyl)phenyl]-3-(trifluoromethyl)pyrazole (B-77);
4-(4-fluorophenyl)-5-[4-(methylsulfonyl)phenyl]-1-phenyl-3-(trifluoromethyl)pyrazole (B-78);
4-(5-(4-chlorophenyl)-3-(4-methoxyphenyl)-1H-pyrazol-1-yl) benzenesulfonamide (B-79);
4-(3,5-bis(4-methylphenyl)-1H-pyrazol-1-yl) benzenesulfonamide (B-80);
4-(5-(4-chlorophenyl)-3-phenyl-1H-pyrazol-1-yl) benzenesulfonamide (B-81);
4-(3,5-bis(4-methoxyphenyl)-1H-pyrazol-1-yl) benzenesulfonamide (B-82);
4-(5-(4-chlorophenyl)-3-(4-methylphenyl)-1H-pyrazol-1-yl) benzenesulfonamide (B-83);
4-(5-(4-chlorophenyl)-3-(4-nitrophenyl)-1H-pyrazol-1-yl) benzenesulfonamide (B-84);
4-(5-(4-chlorophenyl)-3-(5-chloro-2-thienyl)-1H-pyrazol-1-yl) benzenesulfonamide (B-85);
4-(4-chloro-3,5-diphenyl-1H-pyrazol-1-yl)benzenesulfonamide (B-86);
4-[5-(4-chlorophenyl)-3-(trifluoromethyl)-1H-pyrazol-1-yl]benzenesulfonamide (B-87);
4-[5-phenyl-3-(trifluoromethyl)-1H-pyrazol-1-yl]benzenesulfonamide (B-88);
4-[5-(4-fluorophenyl)-3-(trifluoromethyl)-1H-pyrazol-1-yl]benzenesulfonamide (B-89);
4-[5-(4-methoxyphenyl)-3-(trifluoromethyl)-1H-pyrazol-1-yl]benzenesulfonamide (B-90);
4-[5-(4-chlorophenyl)-3-(difluoromethyl)-1H-pyrazol-1-yl]benzenesulfonamide (B-91);
4-[5-(4-methylphenyl)-3-(trifluoromethyl)-1H-pyrazol-1-yl]benzenesulfonamide (B-92);
4-[4-chloro-5-(4-chlorophenyl)-3-(trifluoromethyl)-1H-pyrazol-1-yl]benzenesulfonamide (B-93);
4-[3-(difluoromethyl)-5-(4-methylphenyl)-1H-pyrazol-1-yl]benzenesulfonamide (B-94);
4-[3-(difluoromethyl)-5-phenyl-1H-pyrazol-1-yl]benzenesulfonamide (B-95);
4-[3-(difluoromethyl)-5-(4-methoxyphenyl)-1H-pyrazol-1-yl]benzenesulfonamide (B-96);
4-[3-cyano-5-(4-fluorophenyl)-1H-pyrazol-1-yl]benzenesulfonamide (B-97);
4-[3-(difluoromethyl)-5-(3-fluoro-4-methoxyphenyl)-1H-pyrazol-1-yl]benzenesulfonamide (B-98);
4-[5-(3-fluoro-4-methoxyphenyl)-3-(trifluoromethyl)-1H-pyrazol-1-yl]benzenesulfonamide (B-99);
4-[4-chloro-5-phenyl-1H-pyrazol-1-yl]benzenesulfonamide (B-100);
4-[5-(4-chlorophenyl)-3-(hydroxymethyl)-1H-pyrazol-1-yl]benzenesulfonamide (B-101);
4-[5-(4-(N,N-dimethylamino)phenyl)-3-(trifluoromethyl)-1H-pyrazol-1-yl]benzenesulfonamide (B-102);
5-(4-fluorophenyl)-6-[4-(methylsulfonyl)phenyl]spiro[2.4]hept-5-ene (B-103);
4-[6-(4-fluorophenyl)spiro[2.4]hept-5-en-5-yl]benzenesulfonamide (B-104);
6-(4-fluorophenyl)-7-[4-(methylsulfonyl)phenyl]spiro[3.4]oct-6-ene (B-105);
5-(3-chloro-4-methoxyphenyl)-6-[4-(methylsulfonyl) phenyl]spiro[2.4]hept-5-ene (B-106);
4-[6-(3-chloro-4-methoxyphenyl)spiro[2.4]hept-5-en-5-yl]benzenesulfonamide (B-107);
5-(3,5-dichloro-4-methoxyphenyl)-6-[4-(methylsulfonyl)phenyl]spiro[2.4]hept-5-ene (B-108);
5-(3-chloro-4-fluorophenyl)-6-[4-(methylsulfonyl)phenyl]spiro[2.4]hept-5-ene (B-109);
4-[6-(3,4-dichlorophenyl)spiro[2.4]hept-5-en-5-yl]benzenesulfonamide (B-110);
2-(3-chloro-4-fluorophenyl)-4-(4-fluorophenyl)-5-(4-methylsulfonyl phenyl)thiazole (B-111);
2-(2-chlorophenyl)-4-(4-fluorophenyl)-5-(4-methylsulfonyl phenyl)thiazole (B-112);
5-(4-fluorophenyl)-4-(4-methylsulfonylphenyl)-2-methylthiazole (B-113);
4-(4-fluorophenyl)-5-(4-methylsulfonylphenyl)-2-trifluoromethylthiazole (B-114);
4-(4-fluorophenyl)-5-(4-methylsulfonylphenyl)-2-(2-thienyl)thiazole (B-115);
4-(4-fluorophenyl)-5-(4-methylsulfonylphenyl)-2-benzylaminothiazole (B-116);
4-(4-fluorophenyl)-5-(4-methylsulfonylphenyl)-2-(1-propylamino) thiazole (B-117);
2-[(3,5-dichlorophenoxy)methyl)₄-(4-fluorophenyl)-5-[4-(methyl sulfonyl)phenyl]thiazole (B-118);
5-(4-fluorophenyl)-4-(4-methylsulfonylphenyl)-2-trifluoromethylthiazole (B-119);
1-methylsulfonyl-4-[1,1-dimethyl-4-(4-fluorophenyl)cyclopenta-2,4-dien-3-yl]benzene (B-120);
4-[4-(4-fluorophenyl)-1,1-dimethylcyclopenta-2,4-dien-3-yl]benzenesulfonamide (B-121);
5-(4-fluorophenyl)-6-[4-(methylsulfonyl)phenyl]spiro[2.4]hepta-4,6-diene (B-122);
4-[6-(4-fluorophenyl)spiro[2.4]hepta-4,6-dien-5-yl]benzenesulfonamide (B-123);
6-(4-fluorophenyl)-2-methoxy-5-[4-(methylsulfonyl)phenyl]-pyridine-3-carbonitrile (B-124);
2-bromo-6-(4-fluorophenyl)-5-[4-(methylsulfonyl)phenyl]-pyridine-3-carbonitrile (B-125);
6-(4-fluorophenyl)-5-[4-(methylsulfonyl)phenyl]-2-phenyl-pyridine-3-carbonitrile (B-126);
4-[2-(4-methylpyridin-2-yl)-4-(trifluoromethyl)-1H-imidazol-1-yl]benzenesulfonamide (B-127);
4-[2-(5-methylpyridin-3-yl)-4-(trifluoromethyl)-1H-imidazol-1-yl]benzenesulfonamide (B-128);
4-[2-(2-methylpyridin-3-yl)₄-(trifluoromethyl)-1H-imidazol-1-yl]benzenesulfonamide (B-129);
3-[1-[4-(methylsulfonyl)phenyl]-4-(trifluoromethyl)-1H-imidazol-2-yl]pyridine (B-130);
2-[1-[4-(methylsulfonyl)phenyl-4-(trifluoromethyl)-1H-imidazol-2-yl]pyridine (B-131);
2-methyl-4-[1-[4-(methylsulfonyl)phenyl-4-(trifluoromethyl)-1H-imidazol-2-yl]pyridine (B-132);
2-methyl-6-[1-[4-(methylsulfonyl)phenyl-4-(trifluoromethyl)-1H-imidazol-2-yl]pyridine (B-133);
4-[2-(6-methylpyridin-3-yl)₄-(trifluoromethyl)-1H-imidazol-1-yl]benzenesulfonamide (B-134);
2-(3,4-difluorophenyl)-1-[4-(methylsulfonyl)phenyl]4-(trifluoromethyl)-1H-imidazole (B-135);
4-[2-(4-methylphenyl)-4-(trifluoromethyl)-1H-imidazol-1-yl]benzenesulfonamide (B-136);
2-(4-chlorophenyl)-1-[4-(methylsulfonyl)phenyl]4-methyl-1H-imidazole (B-137);
2-(4-chlorophenyl)-1-[4-(methylsulfonyl)phenyl]4-phenyl-1H-imidazole (B-138);
2-(4-chlorophenyl)₄-(4-fluorophenyl)-1-[4-(methylsulfonyl)phenyl]-1H-imidazole (B-139);
2-(3-fluoro-4-methoxyphenyl)-1-[4-(methylsulfonyl)phenyl-4-(trifluoro methyl)-1H-imidazole (B-140);
1-[4-(methylsulfonyl)phenyl]-2-phenyl-4-trifluoromethyl-1H-imidazole (B-141);
2-(4-methylphenyl)-1-[4-(methylsulfonyl)phenyl]-4-trifluoromethyl-1H-imidazole (B-142);
4-[2-(3-chloro-4-methyl phenyl)-4-(trifluoromethyl)-1H-imidazol-1-yl]benzenesulfonamide (B-143);
2-(3-fluoro-5-methylphenyl)-1-[4-(methylsulfonyl)phenyl]4-(trifluoro methyl)-1H-imidazole (B-144);
4-[2-(3-fluoro-5-methylphenyl)₄-(trifluoromethyl)-1H-imidazol-1-yl]benzenesulfonamide (B-145);
2-(3-methylphenyl)-1-[4-(methylsulfonyl)phenyl]4-trifluoromethyl-1H-imidazole (B-146);
4-[2-(3-methylphenyl)-4-trifluoromethyl-1H-imidazol-1-yl]benzene sulfonamide (B-147);
1-[4-(methylsulfonyl)phenyl]-2-(3-chlorophenyl)₄-trifluoromethyl-1H-imidazole (B-148);
4-[2-(3-chlorophenyl)-4-trifluoromethyl-1H-imidazol-1-yl]benzenesulfonamide (B-149);
4-[2-phenyl-4-trifluoromethyl-1H-imidazol-1-yl]benzenesulfonamide (B-150);
4-[2-(4-methoxy-3-chlorophenyl)₄-trifluoromethyl-1H-imidazol-1-yl]benzenesulfonamide (B-151);
1-allyl-4-(4-fluorophenyl)-3-[4-(methylsulfonyl)phenyl]-5-(trifluoro methyl)-1H-pyrazole (B-152);
4-[1-ethyl-4-(4-fluorophenyl)-5-(trifluoromethyl)-1H-pyrazol-3-yl]benzenesulfonamide (B-153);
N-phenyl-[4-(4-fluorophenyl)-3-[4-(methylsulfonyl)phenyl]-5-(trifluoromethyl)-1H-pyrazol-1-yl]acetamide (B-154);
ethyl [4-(4-fluorophenyl)-3-[4-(methylsulfonyl)phenyl]-5-(trifluoromethyl)-1H-pyrazol-1-yl]acetate (B-155);
4-(4-fluorophenyl)-3-[4-(methylsulfonyl)phenyl]-1-(2-phenylethyl)-1H-pyrazole (B-156);
4-(4-fluorophenyl)-3-[4-(methylsulfonyl)phenyl]-1-(2-phenylethyl)-5-(trifluoromethyl)pyrazole (B-157);
1-ethyl-4-(4-fluorophenyl)-3-[4-(methylsulfonyl)phenyl]-5-(trifluoromethyl)-1H-pyrazole (B-158);
5-(4-fluorophenyl)₄-(4-methylsulfonylphenyl)-2-trifluoromethyl-1H-imidazole (B-159);
4-[4-(methylsulfonyl)phenyl]-5-(2-thiophenyl)-2-(trifluoromethyl)-1H-imidazole (B-160);
5-(4-fluorophenyl)-2-methoxy-4-[4-(methylsulfonyl)phenyl]-6-(trifluoromethyl)pyridine (B-161);
2-ethoxy-5-(4-fluorophenyl)₄-[4-(methylsulfonyl)phenyl]-6-(trifluoromethyl)pyridine (B-162);
5-(4-fluorophenyl)₄-[4-(methylsulfonyl)phenyl]-2-(2-propynyloxy)-6-(trifluoromethyl)pyrid ine (B-163);
2-bromo-5-(4-fluorophenyl)₄-[4-(methylsulfonyl)phenyl]-6-(trifluoromethyl)pyridine (B-164);
4-[2-(3-chloro-4-methoxyphenyl)-4,5-difluorophenyl]benzenesulfonamide (B-165);
1-(4-fluorophenyl)-2-[4-(methylsulfonyl)phenyl]benzene (B-166);
5-difluoromethyl-4-(4-methylsulfonylphenyl)-3-phenylisoxazole (B-167);
4-[3-ethyl-5-phenylisoxazol-4-yl]benzenesulfonamide (B-168);
4-[5-difluoromethyl-3-phenylisoxazol-4-yl]benzenesulfonamide (B-169);
4-[5-hydroxymethyl-3-phenylisoxazol-4-yl]benzenesulfonamide (B-170);
4-[5-methyl-3-phenyl-isoxazol-4-yl]benzenesulfonamide (B-171);
1-[2-(4-fluorophenyl)cyclopenten-1-yl]4-(methylsulfonyl) benzene (B-172);
1-[2-(4-fluoro-2-methylphenyl)cyclopenten-1-yl]4-(methylsulfonyl) benzene (B-173);
1-[2-(4-chlorophenyl)cyclopenten-1-yl]-4-(methylsulfonyl) benzene (B-174);
1-[2-(2,4-dichlorophenyl)cyclopenten-1-yl]-4-(methylsulfonyl) benzene (B-175);
1-[2-(4-trifluoromethylphenyl)cyclopenten-1-yl]4-(methylsulfonyl) benzene (B-176);
1-[2-(4-methylthiophenyl)cyclopenten-1-yl]4-(methyl sulfonyl)benzene (B-177);
1-[2-(4-fluorophenyl)-4,4-dimethylcyclopenten-1-yl]-4-(methylsulfonyl) benzene (B-178);
4-[2-(4-fluorophenyl)4,4-dimethylcyclopenten-1-yl]benzene sulfonamide (B-179);
1-[2-(4-chlorophenyl)4,4-dimethylcyclopenten-1-yl]4-(methylsulfonyl) benzene (B-180);
4-[2-(4-chlorophenyl)-4,4-dimethylcyclopenten-1-yl]benzene sulfonamide (B-181);
4-[2-(4-fluorophenyl)cyclopenten-1-yl]benzenesulfonamide (B-182);
4-[2-(4-chlorophenyl)cyclopenten-1-yl]benzenesulfonamide (B-183);
1-[2-(4-methoxyphenyl)cyclopenten-1-yl]4-(methylsulfonyl) benzene (B-184);
1-[2-(2,3-difluorophenyl)cyclopenten-1-yl]4-(methylsulfonyl) benzene (B-185);
4-[2-(3-fluoro-4-methoxyphenyl)cyclopenten-1-yl]benzenesulfonamide (B-186);
1-[2-(3-chloro-4-methoxyphenyl)cyclopenten-1-yl]4-(methylsulfonyl) benzene (B-187);
4-[2-(3-chloro-4-fluorophenyl)cyclopenten-1-yl]benzenesulfonamide (B-188);
4-[2-(2-methylpyridin-5-yl)cyclopenten-1-yl]benzenesulfonamide (B-189);
ethyl 2-[4-(4-fluorophenyl)-5-[4-(methylsulfonyl) phenyl]oxazol-2-yl]-2-benzyl-acetate (B-190);
2-[4-(4-fluorophenyl)-5-[4-(methylsulfonyl)phenyl]oxazol-2-yl]acetic acid (B-191);
2-(tert-butyl)₄-(4-fluorophenyl)-5-[4-(methylsulfonyl)phenyl]oxazole (B-192);
4-(4-fluorophenyl)-5-[4-(methylsulfonyl)phenyl]-2-phenyloxazole (B-193);
4-(4-fluorophenyl)-2-methyl-5-[4-(methylsulfonyl)phenyl]oxazole (B-194);
4-[5-(3-fluoro-4-methoxyphenyl)-2-trifluoromethyl-4-oxazolyl]benzenesulfonamide (B-195);
6-chloro-7-(1,1-dimethylethyl)-2-trifluoromethyl-2H-1-benzopyran-3-carboxylic acid (B-196);
6-chloro-8-methyl-2-trifluoromethyl-2H-1-benzopyran-3-carboxylic acid (B-197);
5,5-dimethyl-3-(3-fluorophenyl)-4-methylsulfonyl-2(5H)-furanone (B-198);
6-chloro-2-trifluoromethyl-2H-1-benzothiopyran-3-carboxylic acid (B-199);
4-[5-(4-chlorophenyl)-3-(trifluoromethyl)-1H-pyrazol-1-yl]benzene sulfonamide (B-200);
4-[5-(4-methylphenyl)-3-(trifluoromethyl)-1H-pyrazol-1-yl]benzene sulfonamide (B-201);
4-[5-(3-fluoro-4-methoxyphenyl)-3-(difluoromethyl)-1H-pyrazol-1-yl]benzenesulfonamide (B-202);
3-[1-[4-(methylsulfonyl)phenyl]-4-trifluoromethyl-1H-imidazol-2-yl]pyridine (B-203);
2-methyl-5-[1-[4-(methylsulfonyl)phenyl]-4-trifluoromethyl-1H-imidazol-2-yl]pyrid ine (B-204);
4-[2-(5-methylpyridin-3-yl)₄-(trifluoromethyl)-1H-imidazol-1-yl]benzenesulfonamide (B-205);
4-[5-methyl-3-phenylisoxazol-4-yl]benzenesulfonamide (B-206);
4-[5-hydroxymethyl-3-phenylisoxazol-4-yl]benzenesulfonamide (B-207);
[2-trifluoromethyl-5-(3,4-difluorophenyl)-4-oxazolyl]benzenesulfonamide (B-208);
4-[2-methyl-4-phenyl-5-oxazolyl]benzenesulfonamide (B-209);
4-[5-(2-fluoro-4-methoxyphenyl)-2-trifluoromethyl-4-oxazolyl]benzenesulfonamide (B-210);
[2-(2-chloro-6-fluoro-phenylamino)-5-methyl-phenyl]-acetic acid or COX 189 (lumiracoxib; B-211);
N-(4-Nitro-2-phenoxy-phenyl)-methanesulfonamide or nimesulide (B-212);
N-[6-(2,4-difluoro-phenoxy)-1-oxo-indan-5-yl]-methanesulfonamide or flosulide (B-213);
N-[6-(2,4-Difluoro-phenylsulfanyl)-1-oxo-1H-inden-5-yl]-methanesulfonamide, sodium salt or L-745337 (B-214);
N-[5-(4-fluoro-phenylsulfanyl)-thiophen-2-yl]-methanesulfonamide or RWJ-63556 (B-215);
3-(3,4-Difluoro-phenoxy)₄-(4-methanesulfonyl-phenyl)-5-methyl-5-(2,2,2-trifluoro-ethyl)-5H-furan-2-one or L-784512 or L-784512 (B-216);
(5Z)-2-amino-5-[[3,5-bis(1,1-dimethylethyl)₄-hydroxyphenyl]
methylene]4(5H)-thiazolone or darbufelone (B-217);
CS-502 (B-218);
LAS-34475 (B-219);
LAS-34555 (B-220);
S-33516 (B-221);
SD-8381 (B-222);
L-783003 (B-223);
N-[3-(formylamino)₄-oxo-6-phenoxy-4H-1-benzopyran-7-yl]-methanesulfonamide or T-614 (B-224);
D-1367 (B-225);
L-748731 (B-226);
(6aR,10aR)-3-(1,1-dimethylheptyl)-6a,7,10,10a-tetrahydro-1-hydroxy-6,6-dimethyl-6H-d ibenzo[b,d]pyran-9-carboxylic acid or CT3 (B-227);
CGP-28238 (B-228);
4-[[3,5-bis(1,1-dimethylethyl)₄-hydroxyphenyl]methylene]dihydro-2-methyl-2H-1,2-oxazin-3(4H)-one or BF-389 (B-229);
GR-253035 (B-230);
6-dioxo-9H-purin-8-yl-cinnamic acid (B-231);
S-2474 (B-232);
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methyl sulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3
EXAMPLES OF CYCLOOXYGENASE-2 SELECTIVE INHIBITORS AS
EMBODIMENTS

| Compound | |
|---|---|
| Number | Structural Formula |
| B-26 | 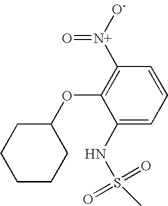 N-(2-cyclohexyloxynitrophenyl)methane sulfonamide or NS-398; |
| B-27 | 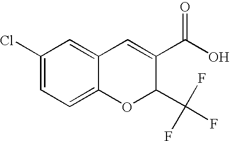 6-chloro-2-trifluoromethyl-2H-1-benzopyran-3-carboxylic acid; |
| B-28 | 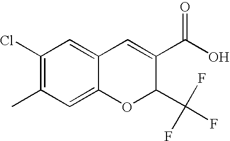 6-chloro-7-methyl-2-trifluoromethyl-2H-1-benzopyran-3-carboxylic acid; |
| B-29 | 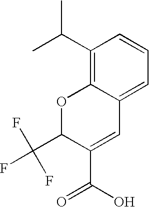 8-(1-methylethyl)-2-trifluoromethyl-2H-1-benzopyran-3-carboxylic acid; |
| B-30 | 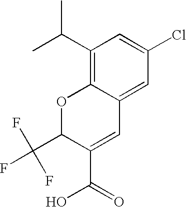 6-chloro-8-(1-methylethyl)-2-trifluoromethyl- 2H-1-benzopyran-3-carboxylic acid; |
| B-31 | 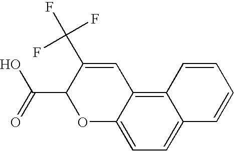 2-trifluoromethyl-3H-naphtho[2,1-b]pyran-3-carboxylic acid; |
| B-32 | 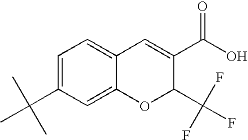 7-(1,1-dimethylethyl)-2-trifluoromethyl-2H-1-benzopyran-3-carboxylic acid; |
| B-33 | 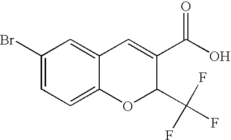 6-bromo-2-trifluoromethyl-2H-1-benzopyran-3-carboxylic acid; |
| B-34 | 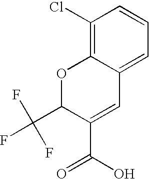 8-chloro-2-trifluoromethyl-2H-1-benzopyran-3-carboxylic acid; |
| B-35 | 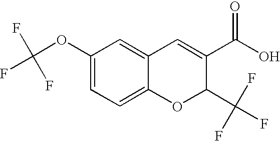 6-trifluoromethoxy-2-trifluoromethyl-2H-1-benzopyran-3-carboxylic acid; |
| B-36 | 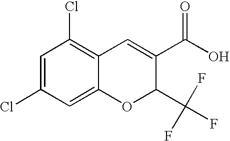 5,7-dichloro-2-trifluoromethyl-2H-1-benzopyran-3-carboxylic acid; |
| B-37 | 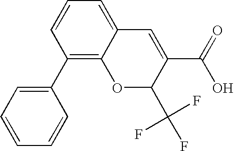 8-phenyl-2-trifluoromethyl-2H-1-benzopyran-3-carboxylic acid; |
| B-38 | 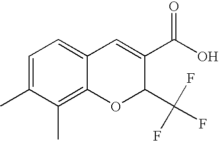 7,8-dimethyl-2-trifluoromethyl-2H-1-benzopyran-3-carboxylic acid; |
| B-39 | 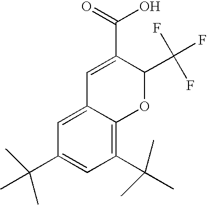 6,8-bis(dimethylethyl)-2-trifluoromethyl-2H-1-benzopyran-3-carboxylic acid; |
| B-40 | 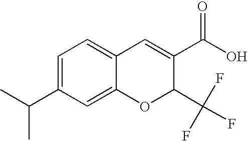 7-(1-methylethyl)-2-trifluoromethyl-2H-1-benzopyran-3-carboxylic acid; |
| B-41 | 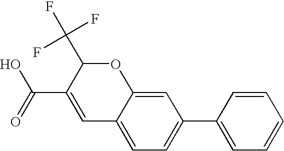 7-phenyl-2-trifluoromethyl-2H-1-benzopyran-3-carboxylic acid; |
| B-42 | 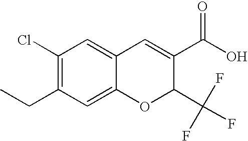 6-chloro-7-ethyl-2-trifluoromethyl-2H-1-benzopyran-3-carboxylic acid; |
| B-43 | 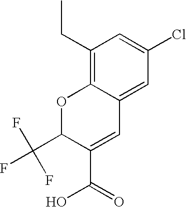 6-chloro-8-ethyl-2-trifluoromethyl-2H-1-benzopyran-3-carboxylic acid; |
| B-44 | 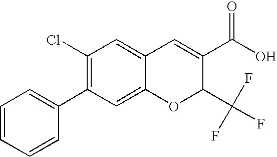 6-chloro-7-phenyl-2-trifluoromethyl-2H-1-benzopyran-3-carboxylic acid; |
| B-45 | 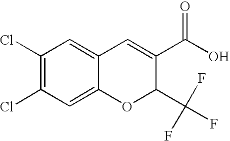 6,7-dichloro-2-trifluoromethyl-2H-1-benzopyran-3-carboxylic acid; |
| B-46 | 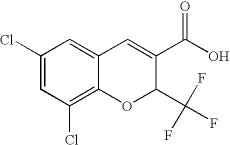 6,8-dichloro-2-trifluoromethyl-2H-1-benzopyran-3-carboxylic acid; |
| B-47 | 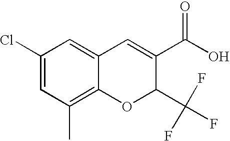 6-chloro-8-methyl-2-trifluoromethyl-2H-1-benzopyran-3-carboxylic acid; |
| B-48 | 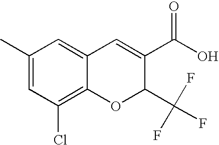 8-chloro-6-methyl-2-trifluoromethyl-2H-1-benzopyran-3-carboxylic acid; |
| B-49 | 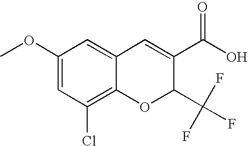 8-chloro-6-methoxy-2-trifluoromethyl-2H-1-benzopyran-3-carboxylic acid; |
| B-50 | 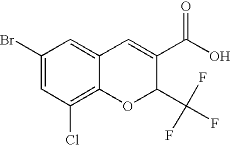 6-bromo-8-chloro-2-trifluoromethyl-2H-1-benzopyran-3-carboxylic acid; |
| B-51 | 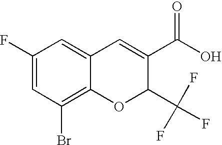 8-bromo-6-fluoro-2-trifluoromethyl-2H-1-benzopyran-3-carboxylic acid; |
| B-52 | 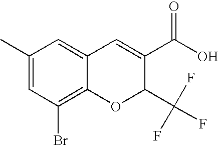 8-bromo-6-methyl-2-trifluoromethyl-2H-1-benzopyran-3-carboxylic acid; |
| B-53 | 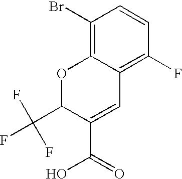 8-bromo-5-fluoro-2-trifluoromethyl-2H-1-benzopyran-3-carboxylic acid; |
| B-54 | 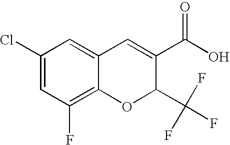 6-chloro-8-fluoro-2-trifluoromethyl-2H-1-benzopyran-3-carboxylic acid; |
| B-55 | 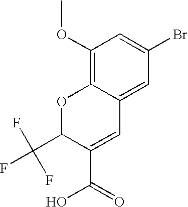 6-bromo-8-methoxy-2-trifluoromethyl-2H-1-benzopyran-3-carboxylic acid; |
| B-56 | 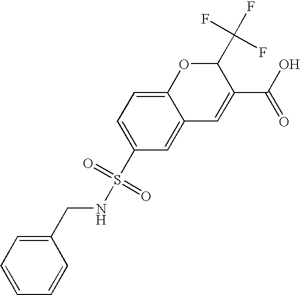 6-[[(phenylmethyl)amino]sulfonyl]-2-trifluoromethyl-2H-1-benzopyran-3-carboxylic acid; |
| B-57 | 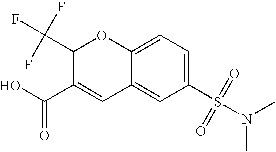 6-[(dimethylamino)sulfonyl]-2-trifluoromethyl-2H-1-benzopyran-3-carboxylic acid; |
| B-58 | 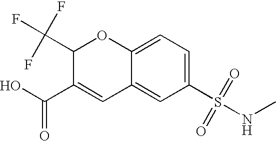 6-[(methylamino)sulfonyl]-2-trifluoromethyl-2H-1-benzopyran-3-carboxylic acid; |
| B-59 | 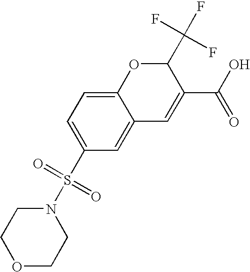 6-[(4-morpholino)sulfonyl]-2-trifluoromethyl-2H-1-benzopyran-3-carboxylic acid; |
| B-60 | 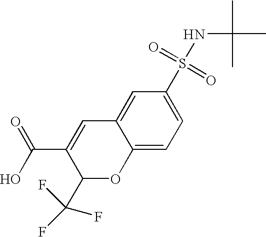 6-[(1,1-dimethylethyl)aminosulfonyl]-2-trifluoromethyl- 2H-1-benzopyran-3-carboxylic acid; |
| B-61 | 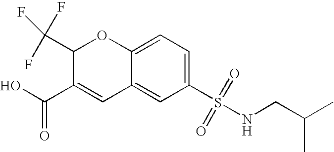 6-[(2-methylpropyl)aminosulfonyl]-2-trifluoromethyl-2H-1-benzopyran-3-carboxylic acid; |
| B-62 | 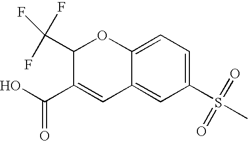 6-methylsulfonyl-2-trifluoromethyl-2H-1-benzopyran-3-carboxylic acid; |
| B-63 | 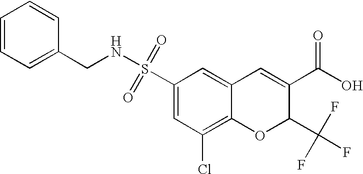 8-chloro-6-[[(phenylmethyl)amino]sulfonyl]-2-trifluoromethyl- 2H-1-benzopyran-3-carboxylic acid; |
| B-64 | 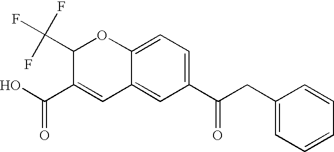 6-phenylacetyl-2-trifluoromethyl-2H-1-benzopyran-3-carboxylic acid; |
| B-65 | 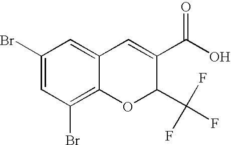 6,8-dibromo-2-trifluoromethyl-2H-1-benzopyran-3-carboxylic acid; |
| B-66 | 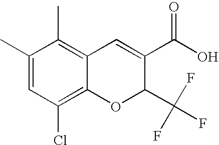 8-chloro-5,6-dimethyl-2-trifluoromethyl-2H-1-benzopyran-3-carboxylic acid; |
| B-67 | 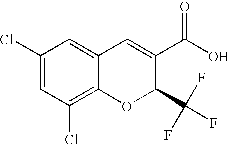 6,8-dichloro-(S)-2-trifluoromethyl-2H-1-benzopyran-3-carboxylic acid; |
| B-68 | 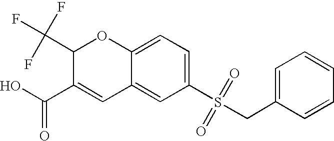 6-benzylsulfonyl-2-trifluoromethyl-2H-1-benzopyran-3-carboxylic acid; |
| B-69 | 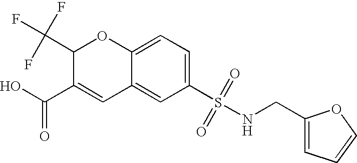 6-[[N-(2-furylmethyl)amino]sulfonyl]-2-trifluoromethyl- 2H-1-benzopyran-3-carboxylic acid; |
| B-70 | 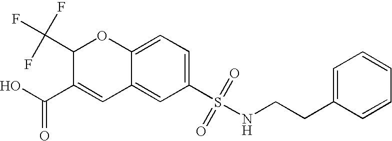 6-[[N-(2-phenylethyl)amino]sulfonyl]-2-trifluoromethyl-2H-1-benzopyran- 3-carboxylic acid; |
| B-71 | 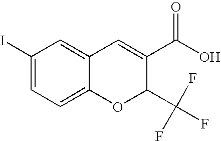 6-iodo-2-trifluoromethyl-2H-1-benzopyran-3-carboxylic acid; |
| B-72 | 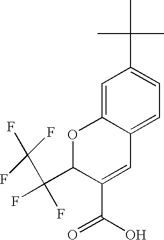 7-(1,1-dimethylethyl)-2-pentafluoroethyl-2H- 1-benzopyran-3-carboxylic acid; |
| B-73 | 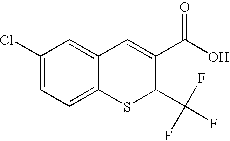 6-chloro-2-trifluoromethyl-2H-1-benzothiopyran-3-carboxylic acid; |
| B-74 | 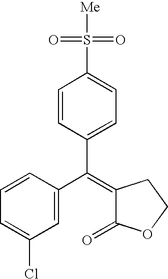 3-[(3-chloro-phenyl)-(4-methanesulfonyl-phenyl)-methylene]- dihydro-furan-2-one or BMS-347070; |
| B-75 | 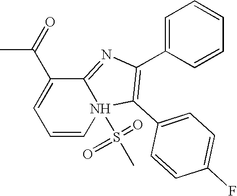 8-acetyl-3-(4-fluorophenyl)-2-(4-methylsulfonyl)phenyl-imidazo(1,2-a)pyridine; |
| B-76 | 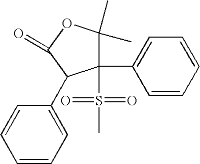 5,5-dimethyl-4-(4-methylsulfonyl)phenyl-3-phenyl-2-(5H)-furanone; |
| B-77 | 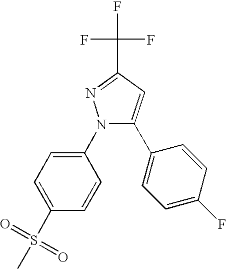 5-(4-fluorophenyl)-1-[4-(methylsulfonyl)phenyl]-3-(trifluoromethyl)pyrazole; |
| B-78 | 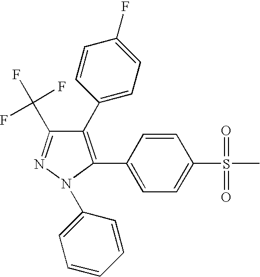 4-(4-fluorophenyl)-5-[4-(methylsulfonyl)phenyl]- 1-phenyl-3-(trifluoromethyl)pyrazole; |
| B-79 | 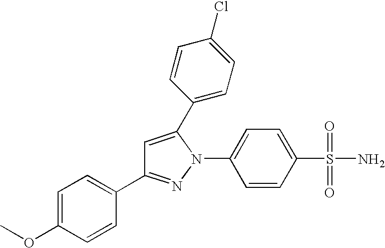 4-(5-(4-chlorophenyl)-3-(4-methoxyphenyl)-1H-pyrazol-1-yl) benzenesulfonamide; |
| B-80 | 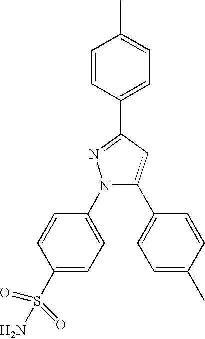 4-(3,5-bis(4-methylphenyl)-1H-pyrazol-1-yl)benzenesulfonamide; |
| B-81 | 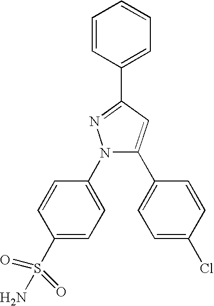 4-(5-(4-chlorophenyl)-3-phenyl-1H-pyrazol-1-yl)benzenesulfonamide; |
| B-82 | 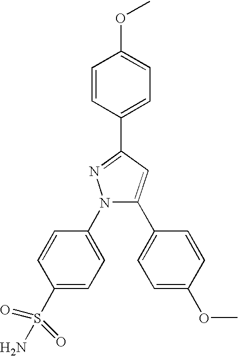 4-(3,5-bis(4-methoxyphenyl)-1H-pyrazol-1-yl)benzenesulfonamide; |
| B-83 | 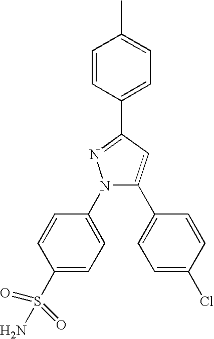 4-(5-(4-chlorophenyl)-3-(4-methylphenyl)-1H-pyrazol-1-yl)benzenesulfonamide; |
| B-84 | 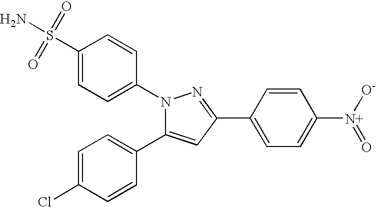 4-(5-(4-chlorophenyl)-3-(4-nitrophenyl)-1H-pyrazol-1-yl)benzenesulfonamide; |
| B-85 | 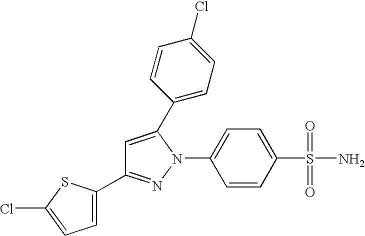 4-(5-(4-chlorophenyl)-3-(5-chloro-2-thienyl)-1H-pyrazol-1-yl)benzenesulfonamide; |
| B-86 | 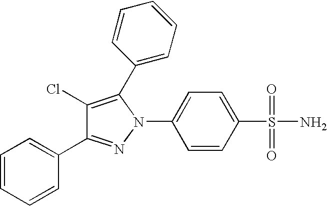 4-(4-chloro-3,5-diphenyl-1H-pyrazol-1-yl)benzenesulfonamide; |
| B-87 | 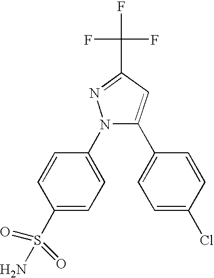 4-[5-(4-chlorophenyl)-3-(trifluoromethyl)-1H-pyrazol-1-yl]benzenesulfonamide; |
| B-88 | 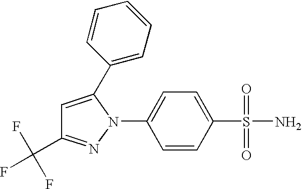 4-[5-phenyl-3-(trifluoromethyl)-1H-pyrazol-1-yl]benzenesulfonamide; |
| B-89 | 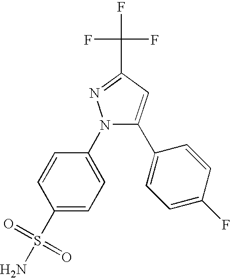 4-[5-(4-fluorophenyl)-3-(trifluoromethyl)-1H-pyrazol-1-yl]benzenesulfonamide; |
| B-90 | 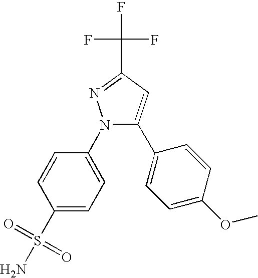 4[5-(4-methoxyphenyl)-3-(trifluoromethyl)-1H-pyrazol-1-yl]benzenesulfonamide; |
| B-91 | 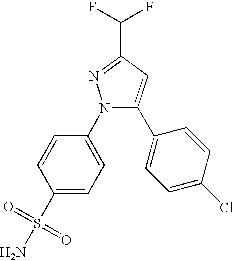 4-[5-(4-chlorophenyl)-3-(difluoromethyl)-1H-pyrazol-1-yl]benzenesulfonamide; |
| B-92 | 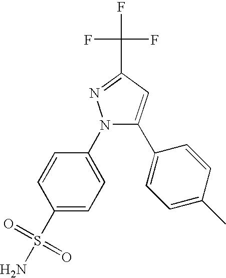 4-[5-(4-methylphenyl)-3-(trifluoromethyl)-1H-pyrazol-1-yl]benzenesulfonamide; |
| B-93 | 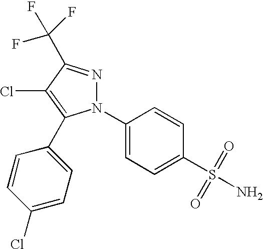 4-[4-chloro-5-(4-chlorophenyl)-3-(trifluoromethyl)-1H-pyrazol-1-yl]benzenesulfonamide; |
| B-94 | 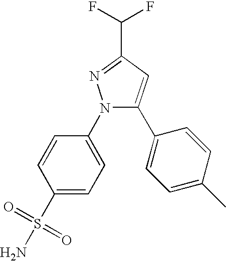 4-[3-(difluoromethyl)-5-(4-methylphenyl)-1H-pyrazol-1-yl]benzenesulfonamide; |
| B-95 | 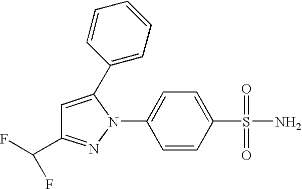 4-[3-(difluoromethyl)-5-phenyl-1H-pyrazol-1-yl]benzenesulfonamide; |
| B-96 | 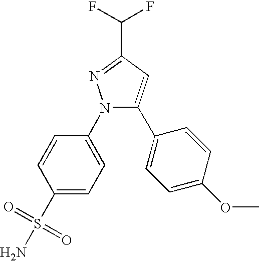 4-[3-(difluoromethyl)-5-(4-methoxyphenyl)-1H-pyrazol-1-yl]benzenesulfonamide; |
| B-97 | 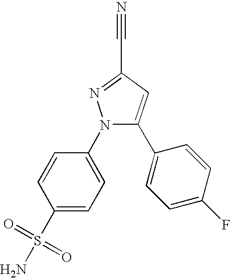 4-[3-cyano-5-(4-fluorophenyl)-1H-pyrazol-1-yl]benzenesulfonamide; |
| B-98 | 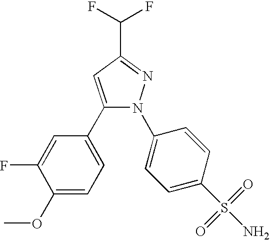 4-[3-(difluoromethyl)-5-(3-fluoro-4-methoxyphenyl)-1H-pyrazol-1-yl]benzenesulfonamide; |
| B-99 | 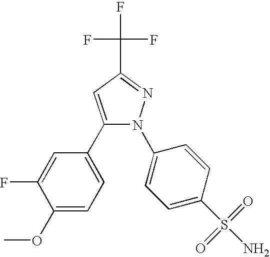 4-[5-(3-fluoro-4-methoxyphenyl)-3-(trifluoromethyl)-1H-pyrazol-1-yl]benzenesulfonamide; |
| B-100 | 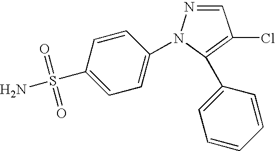 4-[4-chloro-5-phenyl-1H-pyrazol-1-yl]benzenesulfonamide; |
| B-101 | 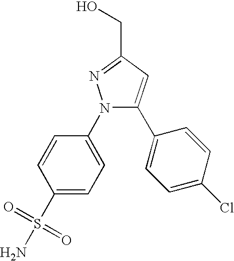 4-[5-(4-chlorophenyl)-3-(hydroxymethyl)-1H-pyrazol-1-yl]benzenesulfonamide; |
| B-102 | 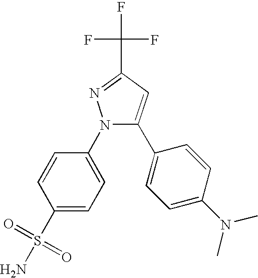 4-[5-(4-(N,N-dimethylamino)phenyl)-3-(trifluoromethyl)- 1H-pyrazol-1-yl]benzenesulfonamide; |
| B-103 | 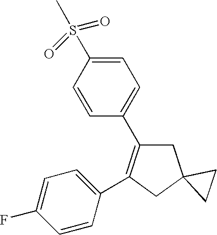 5-(4-fluorophenyl)-6-[4-(methylsulfonyl)phenyl]spiro[2.4]hept-5-ene; |
| B-104 | 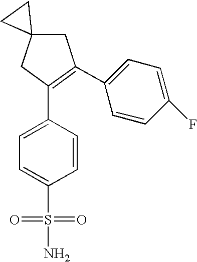 4-[6-(4-fluorophenyl)spiro[2.4]hept-5-en-5-yl]benzenesulfonamide; |
| B-105 | 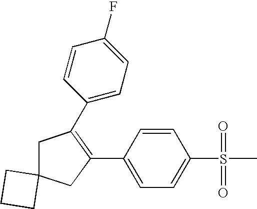 6-(4-fluorophenyl)-7-[4-methylsulfonyl)phenyl]spiro[3.4]oct-6-ene; |
| B-106 | 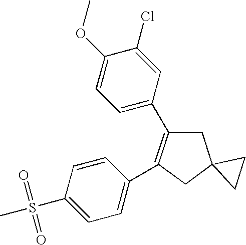 5-(3-chloro-4-methoxyphenyl)-6-[4-(methylsulfonyl)phenyl]spiro[2.4]hept-5-ene; |
| B-107 | 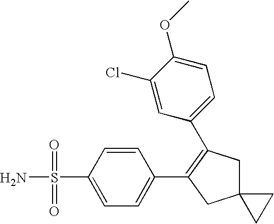 4-[6-(3-chloro-4-methoxyphenyl)spiro[2.4]hept-5-en-5-yl]benzenesulfonamide; |
| B-108 | 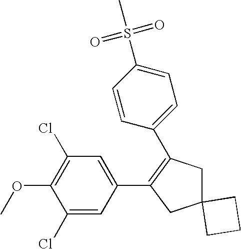 5-(3,5-dichloro-4-methoxyphenyl)-6-[4-(methylsulfonyl)phenyl]spiro[2.4]hept-5-ene; |
| B-109 | 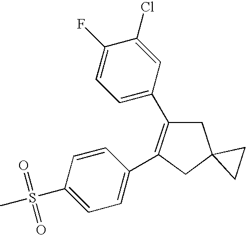 5-(3-chloro-4-fluorophenyl)-6-[4-(methylsulfonyl)phenyl]spiro[2.4]hept-5-ene; |
| B-110 | 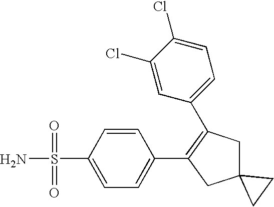 4-[6-(3,4-dichlorophenyl)spiro[2.4]hept-5-en-5-yl]benzenesulfonamide; |
| B-111 | 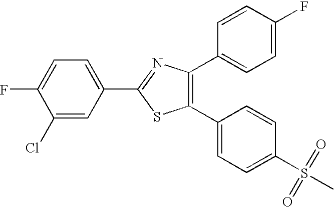 2-(3-chloro-4-fluorophenyl)-4-(4-fluorophenyl)-5-(4-methylsulfonylphenyl)thiazole; |
| B-112 | 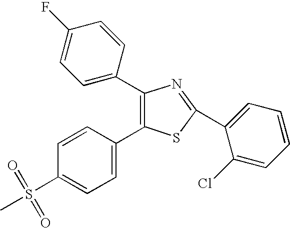 2-(2-chlorophenyl)-4-(4-fluorophenyl)-5-(4-methylsulfonylphenyl)thiazole; |
| B-113 | 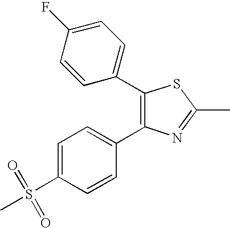 5-(4-fluorophenyl)-4-(4-methylsulfonylphenyl)-2-methylthiazole; |
| B-114 | 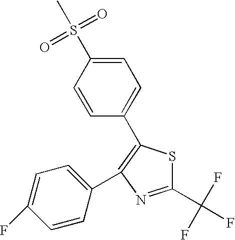 4-(4-fluorophenyl)-5-(4-methylsulfonylphenyl)-2-trifluoromethylthiazole; |
| B-115 | 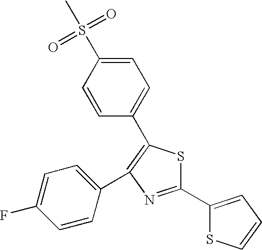 4-(4-fluorophenyl)-5-(4-methylsulfonylphenyl)-2-(2-thienyl)thiazole; |
| B-116 | 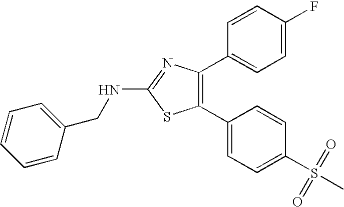 4-(4-fluorophenyl)-5-(4-methylsulfonylphenyl)-2-benzylaminothiazole; |
| B-117 | 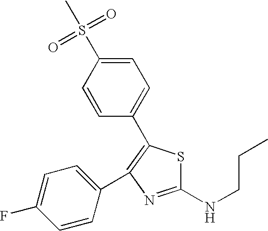 4-(4-fluorophenyl)-5-(4-methylsulfonylphenyl)-2-(1-propylamino)thiazole; |
| B-118 | 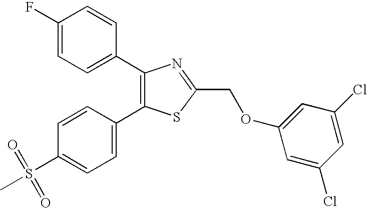 2-((3,5-dichlorophenoxy)methyl)-4-(4-fluorophenyl)-5-[4-(methylsulfonyl)phenyl]thiazole; |
| B-119 | 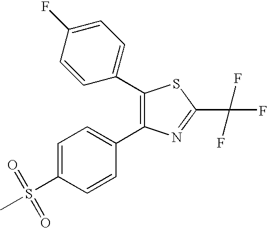 5-(4-fluorophenyl)-4-(4-methylsulfonylphenyl)-2-trifluoromethylthiazole; |
| B-120 | 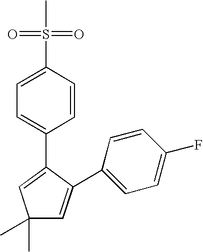 1-methylsulfonyl-4-[1,1-dimethyl-4-(4-fluorophenyl) cyclopenta-2,4-dien-3-yl]benzene; |
| B-121 | 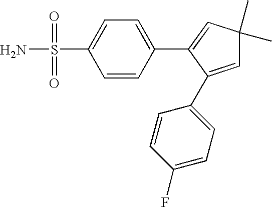 4-[4-(4-fluorophenyl)-1,1-dimethylcyclopenta-2,4-dien-3-yl]benzenesulfonamide; |
| B-122 | 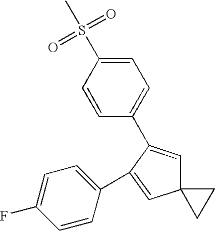 5-(4-fluorophenyl)-6-[4-(methylsulfonyl)phenyl]spiro[2.4]hepta-4,6-diene; |
| B-123 | 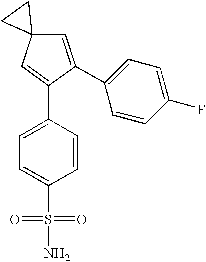 4-[6-(4-fluorophenyl)spiro[2.4]hepta-4,6-dien-5-yl]benzenesulfonamide; |
| B-124 | 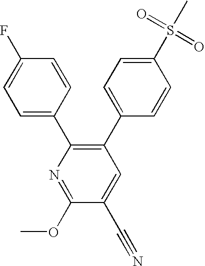 6-(4-fluorophenyl)-2-methoxy-5-[4-(methylsulfonyl)phenyl]- pyridine-3-carbonitrile; |
| B-125 | 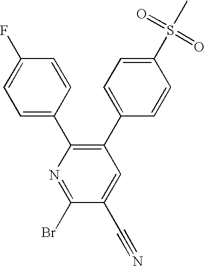 2-bromo-6-(4-fluorophenyl)-5-[4-(methylsulfonyl)phenyl]- pyridine-3-carbonitrile; |
| B-126 | 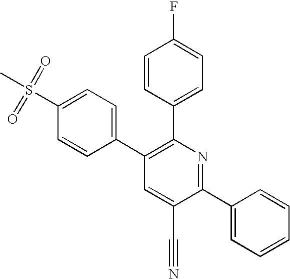 6-(4-fluorophenyl)-5-[4-(methylsulfonyl)phenyl]-2-phenyl-pyridine-3-carbonitrile; |
| B-127 | 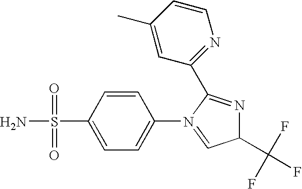 4-[2-(4-methylpyridin-2-yl)-4-(trifluoromethyl)-1H-imidazol-1-yl]benzenesulfonamide; |
| B-128 | 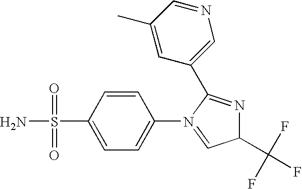 4-[2-(5-methylpyridin-3-yl)-4-(trifluoromethyl)-1H-imidazol-1-yl]benzenesulfonamide; |
| B-129 | 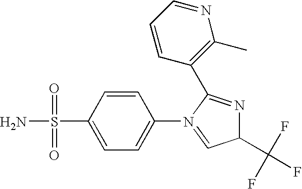 4-[2-(2-methylpyridin-3-yl)-4-(trifluoromethyl)-1H-imidazol-1-yl]benzenesulfonamide; |
| B-130 | 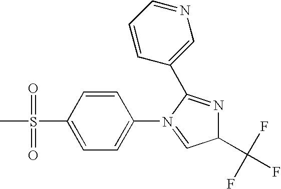 3-[1-[4-(methylsulfonyl)phenyl]-4-(trifluoromethyl)-1H-imidazol-2-yl]pyridine; |
| B-131 | 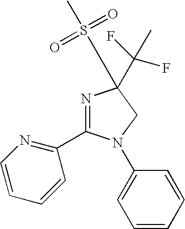 2-[1-[4-(methylsulfonyl)phenyl-4-(trifluoromethyl)]-1H-imidazol-2-yl]pyridine; |
| B-132 | 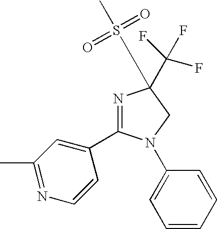 2-methyl-4-[1-[4-(methylsulfonyl)phenyl-4-(trifluoromethyl)]- 1H-imidazol-2-yl]pyridine; |
| B-133 | 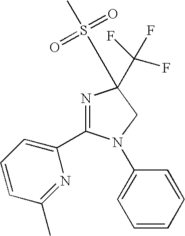 2-methyl-6-[1-[4-(methylsulfonyl)phenyl-4-(trifluoromethyl)]- 1H-imidazol-2-yl]pyridine; |
| B-134 | 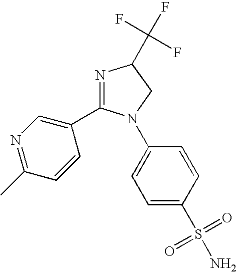 4-[2-(6-methylpyridin-3-yl)-4-(trifluoromethyl)-1H-imidazol-1-yl]benzenesulfonamide; |
| B-135 | 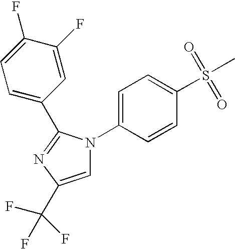 2-(3,4-difluorophenyl)-1-[4-(methylsulfonyl)phenyl]- 4-(trifluoromethyl)-1H-imidazole; |
| B-136 | 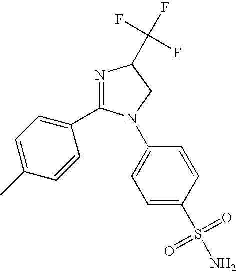 4-[2-(4-methylphenyl)-4-(trifluoromethyl)-1H-imidazol-1-yl]benzenesulfonamide; |
| B-137 | 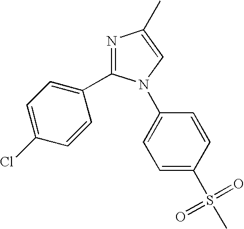 2-(4-chlorophenyl)-1-[4-(methylsulfonyl)phenyl]-4-methyl-1H-imidazole; |
| B-138 | 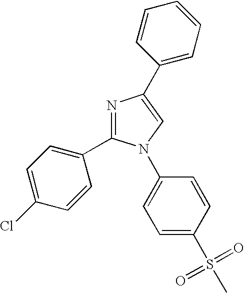 2-(4-chlorophenyl)-1-[4-(methylsulfonyl)phenyl]-4-phenyl-1H-imidazole; |
| B-139 | 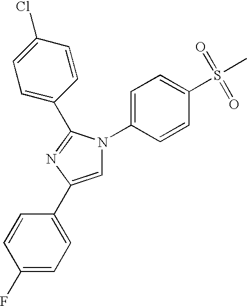 2-(4-chlorophenyl)-4-(4-fluorophenyl)-1-[4-(methylsulfonyl)phenyl]- 1H-imidazole; |
| B-140 | 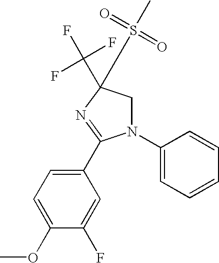 2-(3-fluoro-4-methoxyphenyl)-1-[4-(methylsulfonyl)phenyl- 4-(trifluoromethyl)]-1H-imidazole; |
| B-141 | 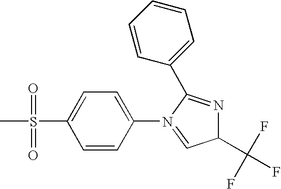 1-[4-(methylsulfonyl)phenyl]-2-phenyl-4-trifluoromethyl-1H-imidazole; |
| B-142 | 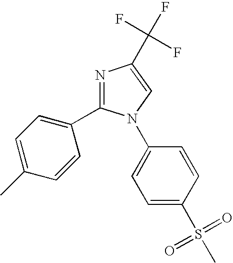 2-(4-methylphenyl)-1-[4-(methylsulfonyl)phenyl]-4-trifluoromethyl-1H-imidazole; |
| B-143 | 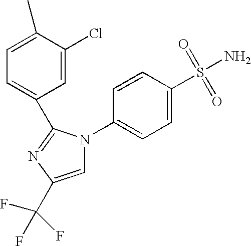 4-[2-(3-chloro-4-methylphenyl)-4-(trifluoromethyl)- 1H-imidazol-1-yl]benzenesulfonamide; |
| B-144 | 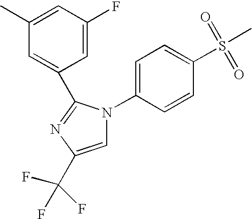 2-(3-fluoro-5-methylphenyl)-1-[4-(methylsulfonyl)phenyl]- 4-(trifluoromethyl)-1H-imidazole; |
| B-145 | 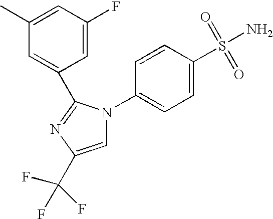 4-[2-(3-fluoro-5-methylphenyl)-4-(trifluoromethyl- 1H-imidazole-1-yl]benzenesulfonamide; |
| B-146 | 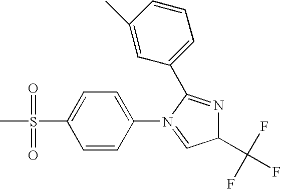 2-(3-methylphenyl)-1-[4-(methylsulfonyl)phenyl]-4-trifluoromethyl-1H-imidazole; |
| B-147 | 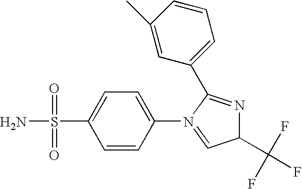 4-[2-(3-methylphenyl)-4-trifluoromethyl-1H-imidazol-1-yl]benzenesulfonamide; |
| B-148 | 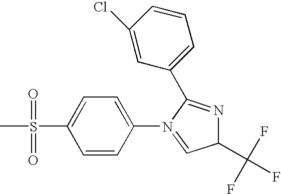 1-[4-(methylsulfonyl)phenyl]-2-(3-chlorophenyl)-4-trifluoromethyl-1H-imidazole |
| B-149 | 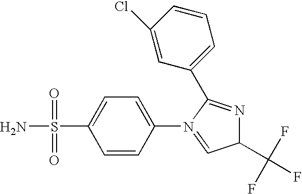 4-[2-(3-chlorophenyl)-4-trifluoromethyl-1H-imidazol-1-yl]benzenesulfonamide; |

| Compound | |
|---|---|
| Number | Structural Formula |
| B-150 | 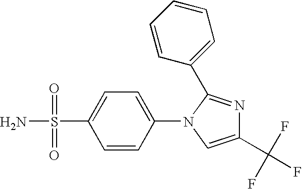 4-[2-phenyl-4-trifluoromethyl-1H-imidazol-1-yl]benzenesulfonamide; |
| B-151 | 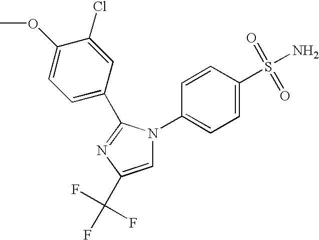 4-[2-(4-methoxy-3-chlorophenyl)-4-trifluoromethyl-1H-imidazol-1-yl]benzenesulfonamide; |
| B-152 | 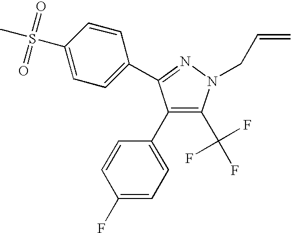 1-allyl-4-(4-fluorophenyl)-3-[4-(methylsulfonyl)phenyl]- 5-(trifluoromethyl)-1H-pyrazole; |
| B-153 | 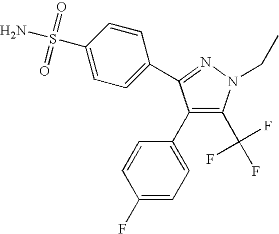 4-[1-ethyl-4-(4-fluorophenyl)-5-(trifluoromethyl)-1H-pyrazol-3-yl]benzenesulfonamide; |
| B-154 | 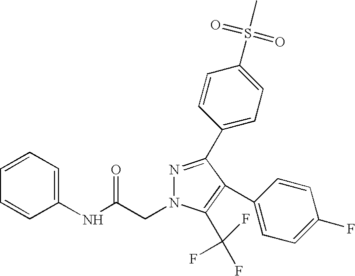 N-phenyl-[4-(4-fluorophenyl)-3-[4-(methylsulfonyl)phenyl]- 5-(trifluoromethyl)-1H-pyrazol-1-yl]acetamide; |
| B-155 | 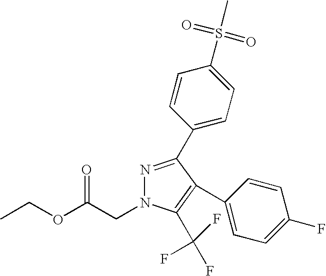 ethyl[4-(4-fluorophenyl)-3-[4-(methylsulfonyl)phenyl]- 5-(trifluoromethyl)-1H-pyrazol-1-yl]acetate; |
| B-156 | 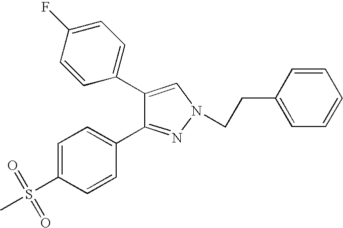 4-(4-fluorophenyl)-3-[4-(methylsulfonyl)phenyl]-1-(2-phenylethyl)-1H-pyrazole; |
| B-157 | 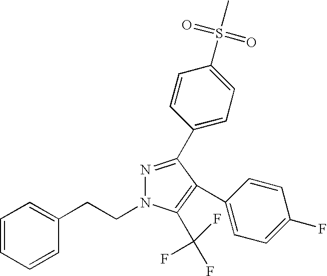 4-(4-fluorophenyl)-3-[4-(methylsulfonyl)phenyl]- 1-(2-phenylethyl)-5-(trifluoromethyl)pyrazole; |
| B-158 | 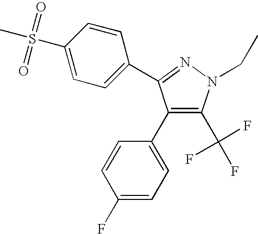 1-ethyl-4-(4-fluorophenyl)-3-[4-methylsulfonyl)phenyl]- 5-(trifluoromethyl)-1H-pyrazole; |
| B-159 | 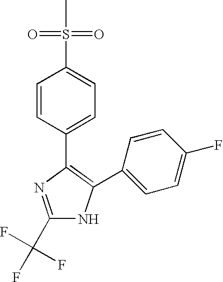 5-(4-fluorophenyl)-4-(4-methylsulfonylphenyl)- 2-trifluoromethyl-1H-imidazole; |
| B-160 | 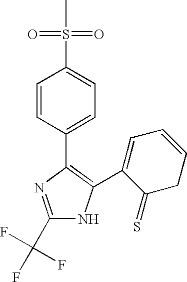 4-[4-(methylsulfonyl)phenyl]-5-(2-thiophenyl)-2-(trifluoromethyl)-1H-imidazole; |
| B-161 | 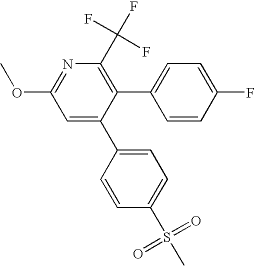 5-(4-fluorophenyl)-2-methoxy-4-[4-(methylsulfonyl)phenyl]-6-(trifluoromethyl)pyridine; |
| B-162 | 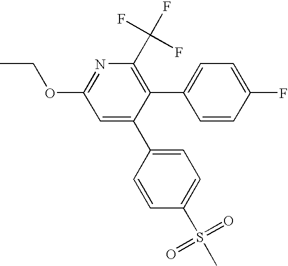 2-ethoxy-5-(4-fluorophenyl)-4-[4-(methylsulfonyl)phenyl]- 6-(trifluoromethyl)pyridine; |
| B-163 | 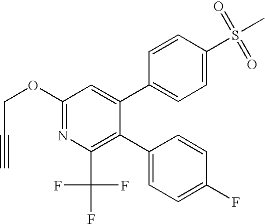 5-(4-fluorophenyl)-4-[4-(methylsulfonyl)phenyl]- 2-(2-propynyloxy)-6-(trifluoromethyl)pyridine; |
| B-164 | 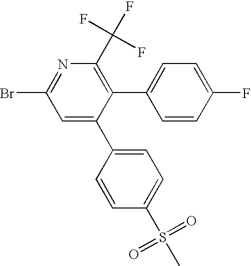 2-bromo-5-(4-fluorophenyl)-4-[4-(methylsulfonyl)phenyl]- 6-(trifluoromethyl)pyridine; |
| B-165 | 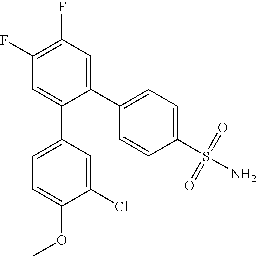 4-[2-(3-chloro-4-methoxyphenyl)-4,5-difluorophenyl]benzenesulfonamide; |
| B-166 | 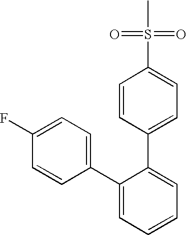 1-(4-fluorophenyl)-2-[4-methylsulfonyl)phenyl]benzene; |
| B-167 | 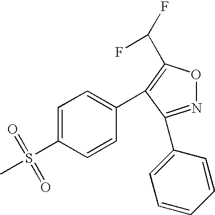 5-difluoromethyl-4-(4-methylsulfonylphenyl)-3-phenylisoxazole; |
| B-168 | 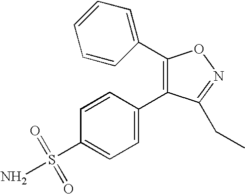 4-[3-ethyl-5-phenylisoxazol-4-yl]benzenesulfonamide; |
| B-169 | 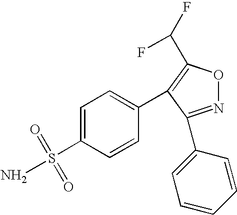 4-[5-difluoromethyl-3-phenylisoxazol-4-yl]benzenesulfonamide; |
| B-170 | 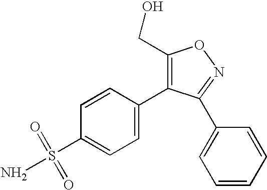 4-[5-hydroxymethyl-3-phenylisoxazol-4-yl]benzenesulfonamide; |
| B-171 | 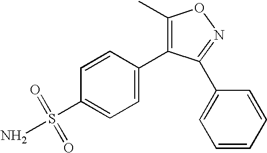 4-[5-methyl-3-phenyl-isoxazol-4-yl]benzenesulfonamide; |
| B-172 | 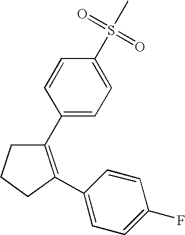 1-[2-(4-fluorophenyl)cyclopenten-1-yl]-4-(methylsulfonyl)benzene; |
| B-173 | 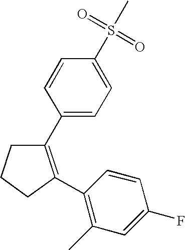 1-[2-(4-fluoro-2-methylphenyl)cyclopenten-1-yl]-4-(methylsulfonyl)benzene; |
| B-174 | 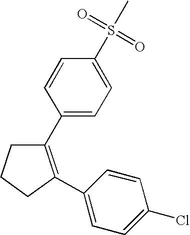 1-[2-(4-chlorophenyl)cyclopenten-1-yl]-4-(methylsulfonyl)benzene; |
| B-175 | 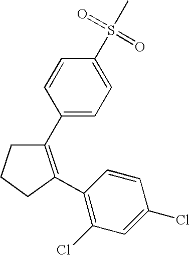 1-[2-(2,4-dichlorophenyl)cyclopenten-1-yl]-4-(methylsulfonyl)benzene; |
| B-176 | 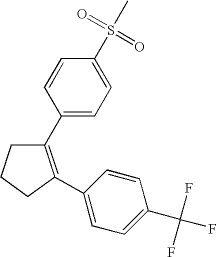 1-[2-(4-trifloromethylphenyl)cyclopenten-1-yl]-4-(methylsulfonyl)benzene; |
| B-177 | 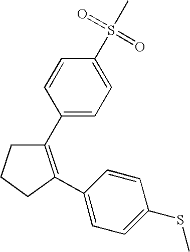 1-[2-(4-methylthiophenyl)cyclopenten-1-yl]-4-(methylsulfonyl)benzene; |
| B-178 | 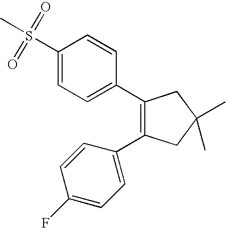 1-[2-(4-fluorophenyl)-4,4-dimethylcyclopenten-1-yl]-4-(methylsulfonyl)benzene; |
| B-179 | 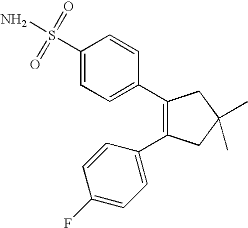 4-[2-(4-fluorophenyl)-4,4-dimethylcyclopenten-1-yl]benzenesulfonamide; |
| B-180 | 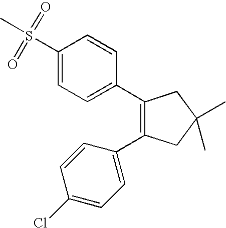 1-[2-(3-chlorophenyl)-4,4-dimethylcyclopenten-1-yl]-4-(methylsulfonyl)benzene; |
| B-181 | 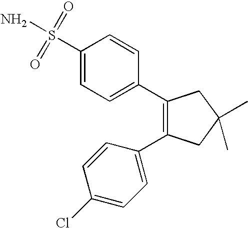 4-[2-(4-chlorophenyl)-4,4-dimethylcyclopenten-1-yl]benzenesulfonamide; |
| B-182 | 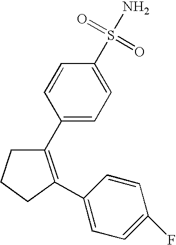 4-[2-(4-fluorophenyl)cyclopenten-1-yl]benzenesulfonamide; |
| B-183 | 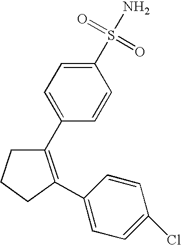 4-[2-(4-chlorophenyl)cyclopenten-1-yl]benzenesulfonamide; |
| B-184 | 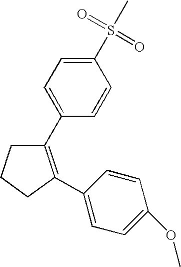 1-[2-(4-methoxyphenyl)cyclopenten-1-yl]-4-(methylsulfonyl)benzene; |
| B-185 | 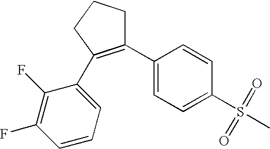 1-[2-(2,3-difluorophenyl)cyclopenten-1-yl]-4-(methylsulfonyl)benzene; |
| B-186 | 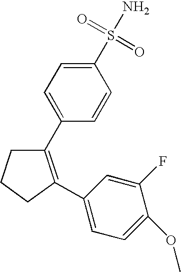 4-[2-(3-fluoro-4-methoxyphenyl)cyclopenten-1-yl]benzenesulfonamide; |
| B-187 | 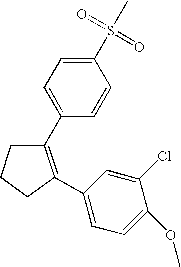 1-[2-(3-chloro-4-methoxyphenyl)cyclopenten-1-yl]-4-(methylsulfonyl)benzene; |
| B-188 | 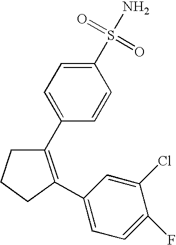 4-[2-(3-chloro-4-fluorophenyl)cyclopenten-1-yl]benzenesulfonamide; |
| B-189 | 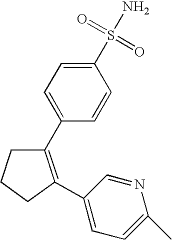 4-[2-(2-methylpyridin-5-yl)cyclopenten-1-yl]benzenesulfonamide; |
| B-190 | 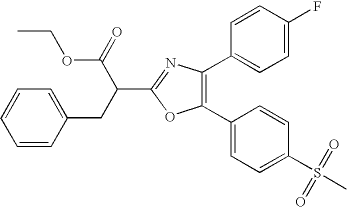 ethyl 2-[4-(4-fluorophenyl)-5-[4-(methylsulfonyl)phenyl]oxazol-2-yl]-2-benzyl-acetate; |
| B-191 | 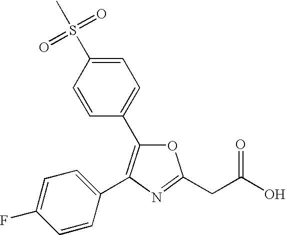 2-[4-(4-fluorophenyl)-5-[4-(methylsulfonyl)phenyl]oxazol-2-yl]acetic acid; |
| B-192 | 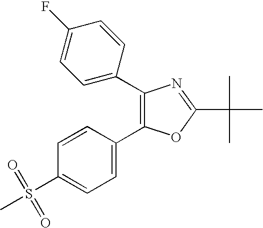 2-(tert-butyl)-4-(4-fluorophenyl)-5-[4-(methylsulfonyl)phenyl]oxazole; |
| B-193 | 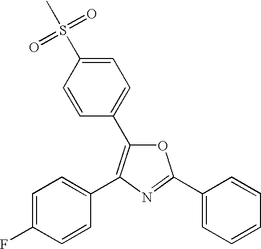 4-(4-fluorophenyl)-5-[4-(methylsulfonyl)phenyl]-2-phenyloxazole; |
| B-194 | 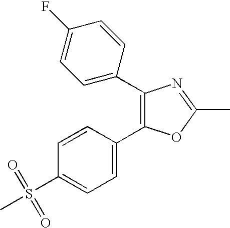 4-(4-fluorophenyl)-2-methyl-5-[4-(methylsulfonyl)phenyl]oxazole; |
| B-195 | 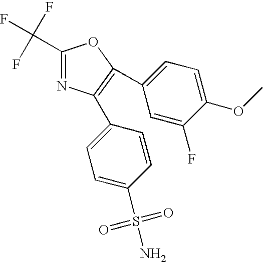 4-[5-(3-fluoro-4-methoxyphenyl)-2-trifluoromethyl- 4-oxazolyl]benzenesulfonamide; |
| B-196 | 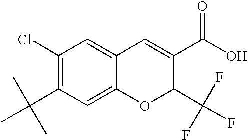 6-chloro-7-(1,1-dimethylethyl)-2-trifluoromethyl-2H- 1-benzopyran-3-carboxylic acid; |
| B-197 | 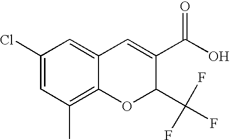 6-chloro-8-methyl-2-trifluoromethyl-2H-1-benzopyran-3-carboxylic acid; |
| B-198 | 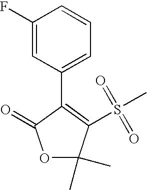 5,5-dimethyl-3-(3-fluorophenyl)-4-methylsulfonyl-2(5H)-furanone; |
| B-199 | 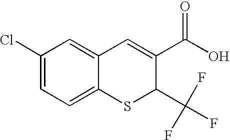 6-chloro-2-trifluoromethyl-2H-1-benzothiopyran-3-carboxyhc acid; |
| B-200 | 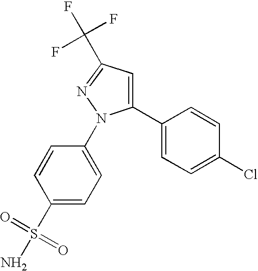 4-[5-(4-chlorophenyl)-3-(trifluoromethyl)-1H-pyrazol-1-yl]benzenesulfonamide; |
| B-201 | 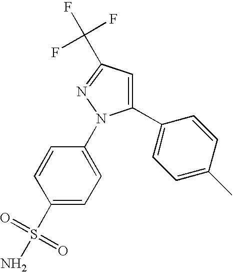 4-[5-(4-methylphenyl)-3-(trifluoromethyl)-1H-pyrazol-1-yl]benzenesulfonamide; |
| B-202 | 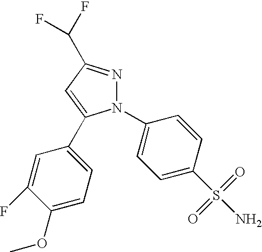 4-[5-(3-fluoro-4-methoxyphenyl)-3-(difluoromethyl)- 1H-pyrazol-1-yl]benzenesulfonamide; |
| B-203 | 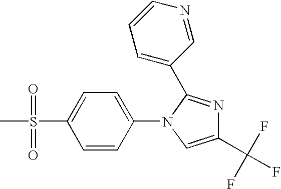 3-[1-[4-(methylsulfonyl)phenyl]-4-trifluoromethyl-1H-imidazol-2-yl]pyridine; |
| B-204 | 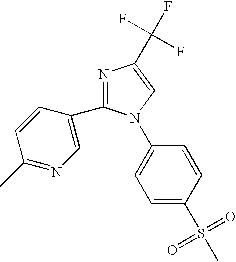 2-methyl-5-[1-[4-(methylsulfonyl)phenyl]-4-trifluoromethyl- 1H-imidazol-2-yl]pyridine; |
| B-205 | 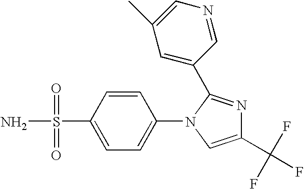 4-[2-(5-methylpyridin-3-yl)-4-(trifluoromethyl)- 1H-imidazol-1-yl]benzenesulfonamide; |
| B-206 | 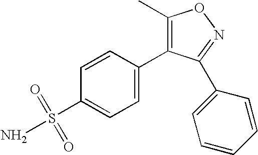 4-[5-methyl-3-phenylisoxazol-4-yl]benzenesulfonamide; |
| B-207 | 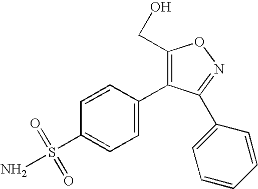 4-[5-hydroxymethyl-3-phenylisoxazol-4-yl]benzenesulfonamide; |
| B-208 | 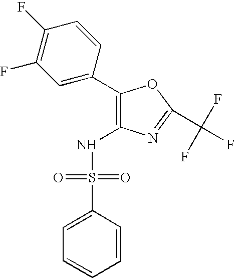 [2-trifluoromethyl-5-(3,4-difluorophenyl)-4-oxazolyl]benzenesulfonamide; |
| B-209 | 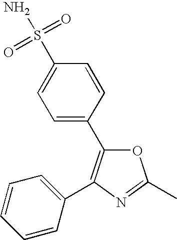 4-[2-methyl-4-phenyl-5-oxazolyl]benzenesulfonamide; |
| B-210 | 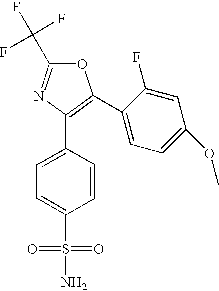 4-[5-(2-fluoro-4-methoxyphenyl)-2-trifluoromethyl-4-oxazolyl]benzenesulfonamide; |
| B-211 | 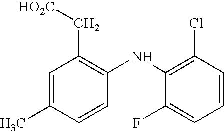 |
| B-212 | 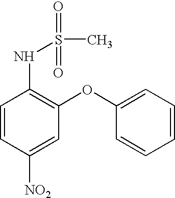 N-(4-nitro-2-phenoxy-phenyl)-methanesulfonamide |
| B-213 | 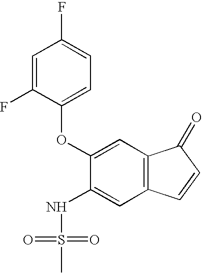 N-[6-(2,4-difluoro-phenoxy)-1-oxo-inden-5-yl]-methanesulfonamide |
| B-214 | 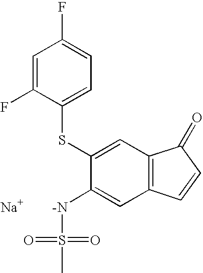 N-[6-(2,4-difluoro-phenylsulfanyl)-1-oxo-1H-inden-5-yl]-methanesulfonamide, soldium salt |
| B-215 | 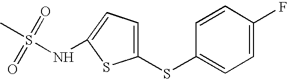 N-[5-(4-fluoro-phenylsulfanyl)-thiophen-2-yl]-methanesulfonamide |
| B-216 | 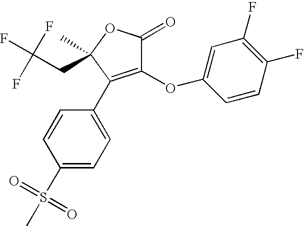 3-(3,4-difluoro-phenoxy)-4-(4-methanesulfonyl-phenyl)-5-methyl- 5-(2,2,2-trifluoro-ethyl)-5H-furan-2-one |
| B-217 | 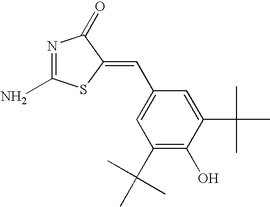 (5Z)-2-amino-5-[[3,5-bis(1,1-dimethylethyl)-4-hydroxyphenyl]methylene]- 4(5H)-thiazolone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 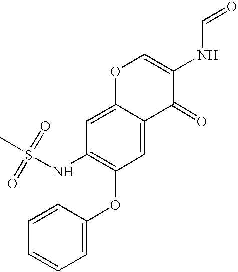 N-[3-(formylamino)-4-oxo-6-phenoxy-4H-1-benzopyran-7-yl]- methanesulfonamide |
| B-225 | D-1367 |
| B-226 | L-748731 |
| B-227 | 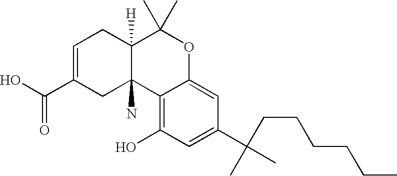 (6aR,10aR)-3-(1,1-dimethylheptyl)-6a,7,10,10a-tetrahydro-1-hydroxy-6,6-dimethyl- 6H-dibenzo[b,d]pyran-9-carboxylic acid |
| B-228 | CGP-28238 |
| B-229 | 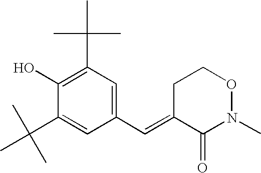 4-[[3,5-bis(1,1-dimethylethyl)-4-hydroxyphenyl]methylene]dihydro-2-methyl-2H-1,2-oxazin-3(4H)-one |
| B-230 | GR-253035 |
| B-231 | 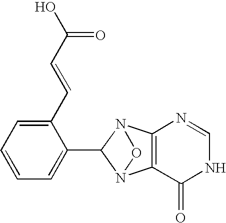 2-(6-dioxo-9H-purin-8-yl)cinnamic acid |
| B-232 | S-2474 |
| B-233 | 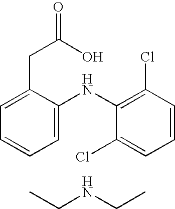 |
| B-234 | 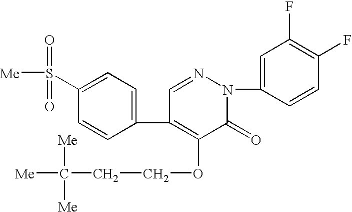 |
| B-235 | 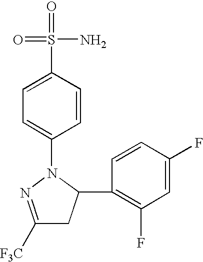 |
| B-236 | 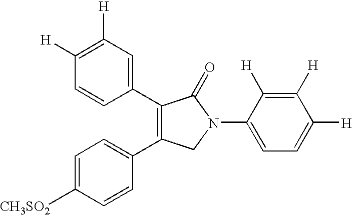 |
| B-237 | 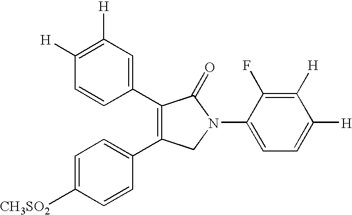 |
| B-238 | 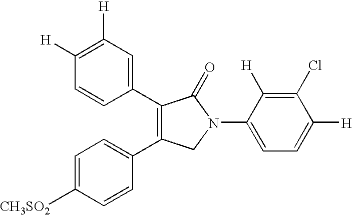 |
| B-239 | 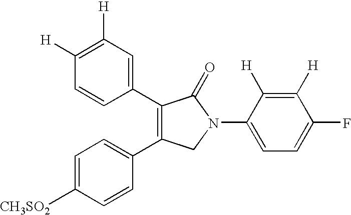 |
| B-240 | 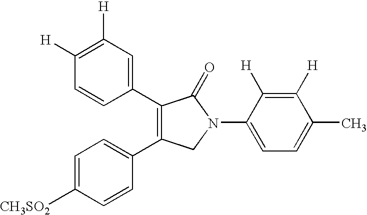 |
| B-241 | 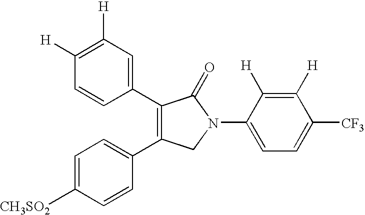 |
| B-242 | 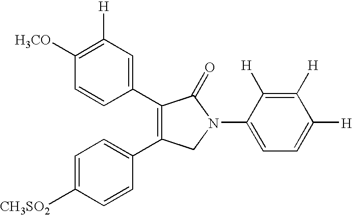 |
| B-243 | 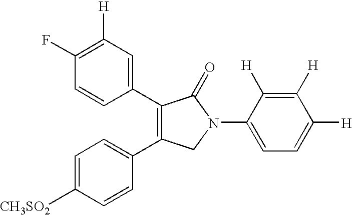 |
| B-244 | 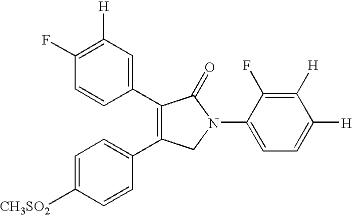 |
| B-245 | 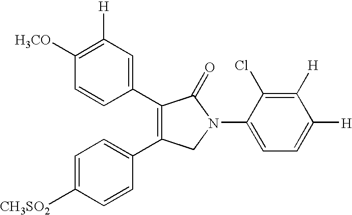 |
| B-246 | 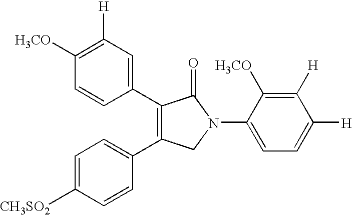 |
| B-247 | 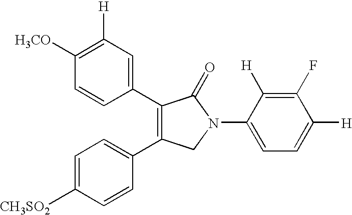 |
| B-248 | 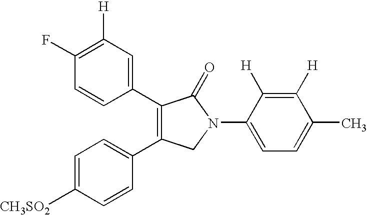 |
| B-249 | 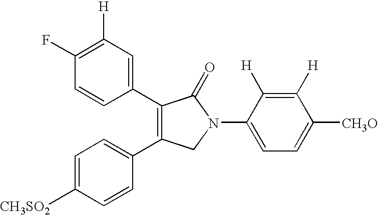 |
| B-250 | 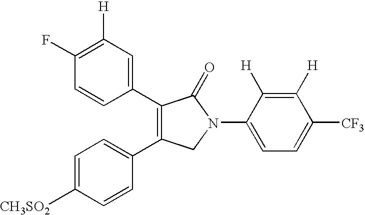 |
| B-251 | 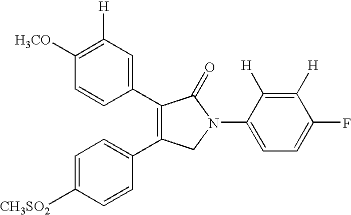 |
| B-252 | 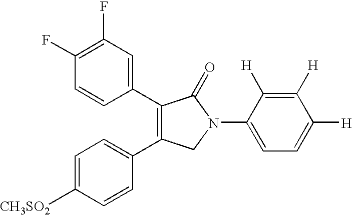 |

The cyclooxygenase-2 selective inhibitor employed in the present invention can exist in tautomeric, geometric or stereoisomeric forms. Generally speaking, suitable cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R- and S-enantiomers, diastereomers, d-isomers, 1-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric•forms. Furthermore, some of the compounds described contain one or more stereocenters and are meant to include R, S, and mixtures or R and S forms for each stereocenter present.

The cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors of the present invention can be formulated into pharmaceutical compositions and administered by a number of different means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose is generally administered in one to about four doses per day.

In one embodiment, when the cyclooxygenase-2 selective inhibitor comprises rofecoxib, it is typical that the amount used is within a range of from about 0.15 to about 1.0 mg/day·kg, and even more typically, from about 0.18 to about 0.4 mg/day kg.

In still another embodiment, when the cyclooxygenase-2 selective inhibitor comprises etoricoxib, it is typical that the amount used is within a range of from about 0.5 to about 5 mg/day·kg, and even more typically, from about 0.8 to about 4 mg/day·kg.

Further, when the cyclooxygenase-2 selective inhibitor comprises celecoxib, it is typical that the amount used is within a range of from about 1 to about 20 mg/day·kg, even more typically, from about 1.4 to about 8.6 mg/day·kg, and yet more typically, from about 2 to about 3 mg/day·kg.

When the cyclooxygenase-2 selective inhibitor comprises valdecoxib, it is typical that the amount used is within a range of from about 0.1 to about 5 mg/day·kg, and even more typically, from about 0.8 to about 4 mg/day kg.

In a further embodiment, when the cyclooxygenase-2 selective inhibitor comprises parecoxib, it is typical that the amount used is within a range of from about 0.1 to about 5 mg/day·kg, and even more typically, from about 1 to about 3 mg/day·kg.

Those skilled in the art will appreciate that dosages may also be determined with guidance from Goodman & Goldman's The Pharmacological Basis of Therapeutics, Ninth Edition (1996), Appendix II, pp.1707-1711 and from Goodman & Goldman's The Pharmacological Basis of Therapeutics, Tenth Edition (2001), Appendix II, pp. 475-493.

Anti-Nausea Agents

In addition to a cyclooxygenase-2 selective inhibitor, the compositions and methods of the invention may also comprise an anti-nausea agent. Several anti-nausea agents can be used in the current invention to the extent that the agent is capable of achieving the desired degree of inhibition of nausea and/or vomiting. A variety of anti-nausea agents are known in the art and include anticholinergics (i.e., scopolamine), antihistamines (i.e., dimenhydrinate, diphenhydramine, hydroxyzine), benzodiazepines (i.e., diazepam, lorazepam), phenothiazines (i.e., chlorpromazine, methotrimeprazine, perphenazine, prochlorperazine, promethazine, trifluoperazine, triflupromazine), benzquinamide, bismuth subsalicylate, buclizine, cinnarizine, cyclizine, diphenidol, dolasetron, domperidone, dronabinol, droperidol, granisetron, haloperidol, metoclopramide, nabilone, ondansetron, thiethylperazine, and trimethobenzamide.

Other agents that can be used to treat nausea or emesis include, for example, NK1 and/or substance P antagonists, opioid modulators or antagonists, and dopamine D4 modulators.

The anti-nausea agents of the present invention can be administered in combination with pharmaceutically acceptable carriers, diluents, adjuvants and vehicles. They can be formulated into pharmaceutical compositions and administered to a subject by any suitable means generally known in the art that will deliver a therapeutically effective dose. For example, these pharmaceutical compositions may be given orally, parenterally, rectally, intradermally, transdermally, or applied topically as an ointment, cream or powder. The usual pharmaceutically acceptable carriers, diluents, adjuvants, vehicles, and additive materials may be used. These may be liquid or solid materials, which are otherwise inert or medically acceptable and are compatible with the active ingredients. Examples of such pharmaceutical adjuvants, diluents, and additive materials, as well as methods of administration include those discussed above for the preparation of pharmaceutical forms of the cyclooxygenase-2 selective inhibitor.

The precise amount of anti-nausea agent for use in the present compositions and methods will vary depending, for example, on the specific drug chosen, or the mode of administration. Generally speaking, the anti-nausea agent can be administered in an amount known to be effective at treating, preventing or inhibiting nausea and/or vomiting. The dosage amounts below are given by way of representative example and actual dosage amounts of the anti-nausea agents may vary and may be less than or greater than these amounts.

In one embodiment, the anti-nausea agent is dimenhydrinate or dramamine or meclizine. The amount of dimenhydrinate for use in the present compositions and methods for an adult dosage is from about 10 mg/dosage to about 50 mg/dosage, wherein the dosage is administered, e.g., from 1 to 5 times a day. More typically, the amount of dimenhydrinate for use in the present compositions or methods is about 50 mg/dosage. In another embodiment, the anti-nausea agent is metoclopramide. The amount of metoclopramide for use in the present compositions and methods for an adult dosage is about 10 mg/dosage.

In one embodiment, the anti-nausea agent is administered to the subject between the time of onset of symptoms of migraine to about 6 hours post onset of symptoms of migraine. In another embodiment, the anti-nausea agent is administered to the subject between the time of onset of symptoms of migraine to about 1 hour post onset of symptoms of migraine.

In yet another embodiment of the invention, the COX-2 selective inhibitor(s) and anti-nausea agent may be administered substantially simultaneously, meaning that both agents may be provided in a single dosage, for example by mixing the agents and incorporating the mixture into a single capsule. Alternatively, the COX-2 selective inhibitor(s) and anti-nausea agent may be administered substantially simultaneously by administration in separate dosages within a short time period, for example within 5 minutes or less. Alternatively, the COX-2 selective inhibitor(s) and anti-nausea agent may be administered sequentially, meaning that separate dosages, and possibly even separate dosage forms of the COX-2 selective inhibitor(s) and anti-nausea agent may be administered at separate times, for example on a staggered schedule but with equal frequency of administration of the COX-2 selective inhibitor(s) and anti-nausea agent. Of course, it is also possible that the COX-2 selective inhibitor(s) may be administered either more or less frequently than the anti-nausea agent. In any case, it is typical that, among successive time periods of a sufficient length, for example one day, the weight ratio of the COX-2 selective inhibitor(s) administered to the weight ratio of anti-nausea agent administered remains constant.

Combination Therapies

Generally speaking, it is contemplated that the composition employed in the practice of the invention may include one or more of any of the cyclooxygenase-2 selective inhibitors detailed above in combination with one or more of any of the anti-nausea agents detailed above. By way of a non-limiting example, Table 4a details a number of suitable combinations that are useful in the methods and compositions of the current invention. The combination may also include an isomer, a pharmaceutically acceptable salt, ester, or prodrug of any of the cyclooxygenase-2 selective inhibitors or anti-nausea agents listed in Table 4a.

TABLE 4a

| Cyclooxygenase-2 Selective | |
|---|---|
| Inhibitor | Anti-nausea agent |
| a compound having formula I | scopolamine |
| a compound having formula I | dimenhydrinate |
| a compound having formula I | diazepam |
| a compound having formula I | lorazepam |
| a compound having formula I | chlorpromazine |
| a compound having formula I | benzquinamide |
| a compound having formula I | buclizine |
| a compound having formula I | diphenidol |
| a compound having formula I | metoclopramide |
| a compound having formula II | scopolamine |
| a compound having formula II | dimenhydrinate |
| a compound having formula II | diazepam |
| a compound having formula II | lorazepam |
| a compound having formula II | chlorpromazine |
| a compound having formula II | benzquinamide |
| a compound having formula II | buclizine |
| a compound having formula II | diphenidol |
| a compound having formula II | metoclopramide |
| a compound having formula III | scopolamine |
| a compound having formula III | dimenhydrinate |
| a compound having formula III | diazepam |
| a compound having formula III | lorazepam |
| a compound having formula III | chlorpromazine |
| a compound having formula III | benzquinamide |
| a compound having formula III | buclizine |
| a compound having formula III | diphenidol |
| a compound having formula III | metoclopramide |
| a compound having formula IV | scopolamine |
| a compound having formula IV | dimenhydrinate |
| a compound having formula IV | diazepam |
| a compound having formula IV | lorazepam |
| a compound having formula IV | chlorpromazine |
| a compound having formula IV | benzquinamide |
| a compound having formula IV | buclizine |
| a compound having formula IV | diphenidol |
| a compound having formula IV | metoclopramide |
| a compound having formula V | scopolamine |
| a compound having formula V | dimenhydrinate |
| a compound having formula V | diazepam |
| a compound having formula V | lorazepam |
| a compound having formula V | chlorpromazine |
| a compound having formula V | benzquinamide |
| a compound having formula V | buclizine |
| a compound having formula V | diphenidol |
| a compound having formula V | metoclopramide |

By way of further example, Table 4b details a number of suitable combinations that may be employed in the methods and compositions of the present invention. The combination may also include an isomer, a pharmaceutically acceptable salt, ester, or prodrug of any of the cyclooxygenase-2 selective inhibitors or anti-nausea agents listed in Table 4b.

TABLE 4b

| Cyclooxygenase-2 Selective Inhibitor | Anti-nausea agent |
|---|---|
| a compound selected from the group consisting | Scopolamine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252. | |
| a compound selected from the group consisting | Dimenhydrinate |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252. | |
| a compound selected from the group consisting | Diazepam |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252. | |
| a compound selected from the group consisting | Lorazepam |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252. | |
| a compound selected from the group consisting | Chlorpromazine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252. | |
| a compound selected from the group consisting | Benzquinamide |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252. | |
| a compound selected from the group consisting | Buclizine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252. | |
| a compound selected from the group consisting | Diphenidol |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252. | |
| a compound selected from the group consisting | Metoclopramide |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, and B-252. | |

By way of yet further example, Table 4c details additional suitable combinations that may be employed in the methods and compositions of the current invention. The combination may also include an isomer, a pharmaceutically acceptable salt, ester, or prodrug of any of the cyclooxygenase-2 selective inhibitors or anti-nausea agents listed in Table 4c.

TABLE 4c

| Cyclooxygenase-2 Selective Inhibitor | Anti-nausea agent |
|---|---|
| Celecoxib | Scopolamine |
| Celecoxib | Dimenhydrinate |
| Celecoxib | Diazepam |
| Celecoxib | Lorazepam |
| Celecoxib | Chlorpromazine |
| Celecoxib | Benzquinamide |
| Celecoxib | Buclizine |
| Celecoxib | Diphenidol |
| Celecoxib | Metoclopramide |
| Deracoxib | Scopolamine |
| Deracoxib | Dimenhydrinate |
| Deracoxib | Diazepam |
| Deracoxib | Lorazepam |
| Deracoxib | Chlorpromazine |
| Deracoxib | Benzquinamide |
| Deracoxib | Buclizine |
| Deracoxib | Diphenidol |
| Deracoxib | Metoclopramide |
| Valdecoxib | Scopolamine |
| Valdecoxib | Dimenhydrinate |
| Valdecoxib | Diazepam |
| Valdecoxib | Lorazepam |
| Valdecoxib | Chlorpromazine |
| Valdecoxib | Benzquinamide |
| Valdecoxib | Buclizine |
| Valdecoxib | Diphenidol |
| Valdecoxib | Metoclopramide |
| Rofecoxib | Scopolamine |
| Rofecoxib | Dimenhydrinate |
| Rofecoxib | Diazepam |
| Rofecoxib | Lorazepam |
| Rofecoxib | Chlorpromazine |
| Rofecoxib | Benzquinamide |
| Rofecoxib | Buclizine |
| Rofecoxib | Diphenidol |
| Rofecoxib | Metoclopramide |
| Etoricoxib | Scopolamine |
| Etoricoxib | Dimenhydrinate |
| Etoricoxib | Diazepam |
| Etoricoxib | Lorazepam |
| Etoricoxib | Chlorpromazine |
| Etoricoxib | Benzquinamide |
| Etoricoxib | Buclizine |
| Etoricoxib | Diphenidol |
| Etoricoxib | Metoclopramide |
| Meloxicam | Scopolamine |
| Meloxicam | Dimenhydrinate |
| Meloxicam | Diazepam |
| Meloxicam | Lorazepam |
| Meloxicam | Chlorpromazine |
| Meloxicam | Benzquinamide |
| Meloxicam | Buclizine |
| Meloxicam | Diphenidol |
| Meloxicam | Metoclopramide |
| Parecoxib | Scopolamine |
| Parecoxib | Dimenhydrinate |
| Parecoxib | Diazepam |
| Parecoxib | Lorazepam |
| Parecoxib | Chlorpromazine |
| Parecoxib | Benzquinamide |
| Parecoxib | Buclizine |
| Parecoxib | Diphenidol |
| Parecoxib | Metoclopramide |
| 4-(4-cyclohexyl-2-methyloxazol-5-yl)-2- | Scopolamine |
| fluorobenzenesulfonamide | |
| 4-(4-cyclohexyl-2-methyloxazol-5-yl)-2- | Dimenhydrinate |
| fluorobenzenesulfonamide | |
| 4-(4-cyclohexyl-2-methyloxazol-5-yl)-2- | Diazepam |
| fluorobenzenesulfonamide | |
| 4-(4-cyclohexyl-2-methyloxazol-5-yl)-2- | Lorazepam |
| fluorobenzenesulfonamide | |
| 4-(4-cyclohexyl-2-methyloxazol-5-yl)-2- | Chlorpromazine |
| fluorobenzenesulfonamide | |
| 4-(4-cyclohexyl-2-methyloxazol-5-yl)-2- | Benzquinamide |
| fluorobenzenesulfonamide | |
| 4-(4-cyclohexyl-2-methyloxazol-5-yl)-2- | Buclizine |
| fluorobenzenesulfonamide | |
| 4-(4-cyclohexyl-2-methyloxazol-5-yl)-2- | Diphenidol |
| fluorobenzenesulfonamide | |
| 4-(4-cyclohexyl-2-methyloxazol-5-yl)-2- | Metoclopramide |
| fluorobenzenesulfonamide | |
| 2-(3,5-difluorophenyl)-3-(4- | Scopolamine |
| (methylsulfonyl)phenyl)-2-cyclopenten-1- | |
| one | |
| 2-(3,5-difluorophenyl)-3-(4- | Dimenhydrinate |
| (methylsulfonyl)phenyl)-2-cyclopenten-1- | |
| one | |
| 2-(3,5-difluorophenyl)-3-(4- | Diazepam |
| (methylsulfonyl)phenyl)-2-cyclopenten-1- | |
| one | |
| 2-(3,5-difluorophenyl)-3-(4- | Lorazepam |
| (methylsulfonyl)phenyl)-2-cyclopenten-1- | |
| one | |
| 2-(3,5-difluorophenyl)-3-(4- | Chlorpromazine |
| (methylsulfonyl)phenyl)-2-cyclopenten-1- | |
| one | |
| 2-(3,5-difluorophenyl)-3-(4- | Benzquinamide |
| (methylsulfonyl)phenyl)-2-cyclopenten-1- | |
| one | |
| 2-(3,5-difluorophenyl)-3-(4- | Buclizine |
| (methylsulfonyl)phenyl)-2-cyclopenten-1- | |
| one | |
| 2-(3,5-difluorophenyl)-3-(4- | Diphenidol |
| (methylsulfonyl)phenyl)-2-cyclopenten-1- | |
| one | |
| 2-(3,5-difluorophenyl)-3-(4- | Metoclopramide |
| (methylsulfonyl)phenyl)-2-cyclopenten-1- | |
| one | |
| N-[2-(cyclohexyloxy)-4- | Scopolamine |
| nitrophenyl]methanesulfonamide | |
| N-[2-(cyclohexyloxy)-4- | Dimenhydrinate |
| nitrophenyl]methanesulfonamide | |
| N-[2-(cyclohexyloxy)-4- | Diazepam |
| nitrophenyl]methanesulfonamide | |
| N-[2-(cyclohexyloxy)-4- | Lorazepam |
| nitrophenyl]methanesulfonamide | |
| N-[2-(cyclohexyloxy)-4- | Chlorpromazine |
| nitrophenyl]methanesulfonamide | |
| N-[2-(cyclohexyloxy)-4- | Benzquinamide |
| nitrophenyl]methanesulfonamide | |
| N-[2-(cyclohexyloxy)-4- | Buclizine |
| nitrophenyl]methanesulfonamide | |
| N-[2-(cyclohexyloxy)-4- | Diphenidol |
| nitrophenyl]methanesulfonamide | |
| N-[2-(cyclohexyloxy)-4- | Metoclopramide |
| nitrophenyl]methanesulfonamide | |
| 2-(3,4-difluorophenyl)-4-(3-hydroxy-3- | Scopolamine |
| methylbutoxy)-5-[4- | |
| (methylsulfonyl)phenyl]-3(2H)- | |
| pyridazinone | |
| 2-(3,4-difluorophenyl)-4-(3-hydroxy-3- | Dimenhydrinate |
| methylbutoxy)-5-[4- | |
| (methylsulfonyl)phenyl]-3(2H)- | |
| pyridazinone | |
| 2-(3,4-difluorophenyl)-4-(3-hydroxy-3- | Diazepam |
| methylbutoxy)-5-[4- | |
| (methylsulfonyl)phenyl]-3(2H)- | |
| pyridazinone | |
| 2-(3,4-difluorophenyl)-4-(3-hydroxy-3- | Lorazepam |
| methylbutoxy)-5-[4- | |
| (methylsulfonyl)phenyl]-3(2H)- | |
| pyridazinone | |
| 2-(3,4-difluorophenyl)-4-(3-hydroxy-3- | Chlorpromazine |
| methylbutoxy)-5-[4- | |
| (methylsulfonyl)phenyl]-3(2H)- | |
| pyridazinone | |
| 2-(3,4-difluorophenyl)-4-(3-hydroxy-3- | Benzquinamide |
| methylbutoxy)-5-[4- | |
| (methylsulfonyl)phenyl]-3(2H)- | |
| pyridazinone | |
| 2-(3,4-difluorophenyl)-4-(3-hydroxy-3- | Buclizine |
| methylbutoxy)-5-[4- | |
| (methylsulfonyl)phenyl]-3(2H)- | |
| pyridazinone | |
| 2-(3,4-difluorophenyl)-4-(3-hydroxy-3- | Diphenidol |
| methylbutoxy)-5-[4- | |
| (methylsulfonyl)phenyl]-3(2H)- | |
| pyridazinone | |
| 2-(3,4-difluorophenyl)-4-(3-hydroxy-3- | Metoclopramide |
| methylbutoxy)-5-[4- | |
| (methylsulfonyl)phenyl]-3(2H)- | |
| pyridazinone | |
| 2-[(2,4-dichloro-6-methylphenyl)amino]-5- | Scopolamine |
| ethyl-benzeneacetic acid | |
| 2-[(2,4-dichloro-6-methylphenyl)amino]-5- | Dimenhydrinate |
| ethyl-benzeneacetic acid | |
| 2-[(2,4-dichloro-6-methylphenyl)amino]-5- | Diazepam |
| ethyl-benzeneacetic acid | |
| 2-[(2,4-dichloro-6-methylphenyl)amino]-5- | Lorazepam |
| ethyl-benzeneacetic acid | |
| 2-[(2,4-dichloro-6-methylphenyl)amino]-5- | Chlorpromazine |
| ethyl-benzeneacetic acid | |
| 2-[(2,4-dichloro-6-methylphenyl)amino]-5- | Benzquinamide |
| ethyl-benzeneacetic acid | |
| 2-[(2,4-dichloro-6-methylphenyl)amino]-5- | Buclizine |
| ethyl-benzeneacetic acid | |
| 2-[(2,4-dichloro-6-methylphenyl)amino]-5- | Diphenidol |
| ethyl-benzeneacetic acid | |
| 2-[(2,4-dichloro-6-methylphenyl)amino]-5- | Metoclopramide |
| ethyl-benzeneacetic acid | |
| (3Z)-3-[(4-chlorophenyl)[4- | Scopolamine |
| (methylsulfonyl)phenyl]methylene]dihydro- | |
| 2(3H)-furanone | |
| (3Z)-3-[(4-chlorophenyl)[4- | Dimenhydrinate |
| (methylsulfonyl)phenyl]methylene]dihydro- | |
| 2(3H)-furanone | |
| (3Z)-3-[(4-chlorophenyl)[4- | Diazepam |
| (methylsulfonyl)phenyl]methylene]dihydro- | |
| 2(3H)-furanone | |
| (3Z)-3-[(4-chlorophenyl)[4- | Lorazepam |
| (methylsulfonyl)phenyl]methylene]dihydro- | |
| 2(3H)-furanone | |
| (3Z)-3-[(4-chlorophenyl)[4- | Chlorpromazine |
| (methylsulfonyl)phenyl]methylene]dihydro- | |
| 2(3H)-furanone | |
| (3Z)-3-[(4-chlorophenyl)[4- | Benzquinamide |
| (methylsulfonyl)phenyl]methylene]dihydro- | |
| 2(3H)-furanone | |
| (3Z)-3-[(4-chlorophenyl)[4- | Buclizine |
| (methylsulfonyl)phenyl]methylene]dihydro- | |
| 2(3H)-furanone | |
| (3Z)-3-[(4-chlorophenyl)[4- | Diphenidol |
| (methylsulfonyl)phenyl]methylene]dihydro- | |
| 2(3H)-furanone | |
| (3Z)-3-[(4-chlorophenyl)[4- | Metoclopramide |
| (methylsulfonyl)phenyl]methylene]dihydro- | |
| 2(3H)-furanone | |
| (S)-6,8-dichloro-2-(trifluoromethyl)-2H-1- | Scopolamine |
| benzopyran-3-carboxylic acid | |
| (S)-6,8-dichloro-2-(trifluoromethyl)-2H-1- | Dimenhydrinate |
| benzopyran-3-carboxylic acid | |
| (S)-6,8-dichloro-2-(trifluoromethyl)-2H-1- | Diazepam |
| benzopyran-3-carboxylic acid | |
| (S)-6,8-dichloro-2-(trifluoromethyl)-2H-1- | Lorazepam |
| benzopyran-3-carboxylic acid | |
| (S)-6,8-dichloro-2-(trifluoromethyl)-2H-1- | Chlorpromazine |
| benzopyran-3-carboxylic acid | |
| (S)-6,8-dichloro-2-(trifluoromethyl)-2H-1- | Benzquinamide |
| benzopyran-3-carboxylic acid | |
| (S)-6,8-dichloro-2-(trifluoromethyl)-2H-1- | Buclizine |
| benzopyran-3-carboxylic acid | |
| (S)-6,8-dichloro-2-(trifluoromethyl)-2H-1- | Diphenidol |
| benzopyran-3-carboxylic acid | |
| (S)-6,8-dichloro-2-(trifluoromethyl)-2H-1- | Metoclopramide |
| benzopyran-3-carboxylic acid | |
| Lumiracoxib | Scopolamine |
| Lumiracoxib | Dimenhydrinate |
| Lumiracoxib | Diazepam |
| Lumiracoxib | Lorazepam |
| Lumiracoxib | Chlorpromazine |
| Lumiracoxib | Benzquinamide |
| Lumiracoxib | Buclizine |
| Lumiracoxib | Diphenidol |
| Lumiracoxib | Metoclopramide |

Diagnosis and Indications to be Treated

One aspect of the invention encompasses diagnosing a subject in need of treatment or prevention of a migraine accompanied by nausea.

A migraine can be diagnosed by determining whether a subject has distinguishing migraine features. These distinguishing features were established by the International Headache Society (IHS) and were introduced for both migraines with and without aura (formerly known as “classic” and “common” migraine, respectively). Tables 5a and 5b contain diagnostic criteria for migraine with or without aura as described by Headache Classification Committee of the International Headache Society in Classification and diagnostic criteria for headache disorders, cranial neuralgias and facial pain, Cephalalgia, 1988; 8 (Suppl 7):1-96.

TABLE 5a
INTERNATIONAL HEADACHE SOCIETY CRITERIA
FOR MIGRAINE WITHOUT AURA

```
A. At least 5 attacks that fulfill criteria in B, C, and D
B. Headache attacks that last 4 to 72 hrs (untreated or unsuccessfully
  treated)
C. Headache has at least 2 of the following characteristics:
      1. Unilateral site
      2. Pulsating quality
      3. Moderate to severe intensity
      4. Aggravation by walking stairs or similar routine physical
        activity
D. During headache, at least 1 of the followign symptoms:
      1. Nausea or vomiting (or both)
      2. Photophobia and phonophobia
      3. No evidence of related organic disease
```

TABLE 5b
INTERNATIONAL HEADACHE SOCIETY CRITERIA
FOR MIGRAINE WITH AURA

```
A. At least 2 attacks that fulfill criteria in B and C
B. At least 3 of the following 4 characteristics:
      1. One or more completely reversible aura symptoms that
        indicate focal cerebral cortical or brain-stem dysfunction (or
        both)
      2. At least one aura symptom develops gradually over >4 min
        or two or more symptoms occur in succession
      3. No aura symptom lasts >60 min
      4. Headache follows aura in <1 hr
C. No evidence of related organic disease
```

It should be noted that in addition to the criteria identified above, a number of physicians use other criteria known in the art for diagnosis of migraine. Thus, methods others than the one noted above can be used to make a migraine diagnosis.

Once it is established that a subject suffers from migraine headaches, it should be noted whether the migraines are accompanied by nausea and/or vomiting. If the subject experiences either or both of these symptoms during a migraine, he can be treated as described herein.

Typically, the composition comprising a therapeutically effective amount of a cyclooxygenase-2 selective inhibitor and a therapeutically effective amount of an anti-nausea agent may be employed to treat a migraine accompanied by nausea or vomiting. In one embodiment, the composition can be administered once the subject experiences nausea or vomiting. Alternatively, in subjects who frequently experience nausea or vomiting associated with migraine, the administration of the present combination can be started at the onset of the first migraine symptoms or within 6 hours of the onset of the migraine symptoms.

### EXAMPLES

The following examples are intended to provide illustrations of the application of the present invention. The following examples are not intended to completely define or otherwise limit the scope of the invention.

In the examples below, a combination therapy contains an anti-nausea agent, such as a scopolamine and a COX-2 selective inhibitor. The efficacy of such combination therapy can be evaluated in comparison to a control treatment such as a placebo treatment, administration of a COX-2 selective inhibitor only, or administration of an anti-nausea agent only. By way of example, a combination therapy may contain scopolamine and celecoxib, meclizine and valdecoxib, lorazepam and rofecoxib, or diazepam and celecoxib. It should be noted that these are only several examples, and that any of the anti-nausea agents and COX-2 selective inhibitors of the present invention may be tested as a combination therapy. The dosages of the anti-nausea agent and COX-2 selective inhibitor in a particular therapeutic combination may be readily determined by a skilled artisan conducting the study. The length of the study treatment will vary on a particular study and can also be determined by one of ordinary skill in the art. By way of example, the combination therapy may be administered for the duration of a migraine headache. The anti-nausea agent and COX-2 selective inhibitor can be administered by any route as described herein, but are preferably administered orally for human subjects.

### Example 1

### Evaluation OF COX-1 and COX-2 Activity In Vitro

The COX-2 selective inhibitors suitable for use in this invention exhibit selective inhibition of COX-2 over COX-1 when tested in vitro according to the following activity assays.

Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10⁷-10⁸ pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are preincubated with the enzyme for 10-20 minutes prior to the addition of arachidonic acid. Any reaction between the arachidonic acid and the enzyme is stopped after ten minutes at 37° C. by transferring 40 μl of reaction mix into 160 μl ELISA buffer and 25 μM indomethacin. The PGE2 formed is measured by standard ELISA technology (Cayman Chemical).

Fast Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C. by transferring 40 μl of reaction mix into 160 μl ELISA buffer and 25 μM indomethacin. Indomethacin, a non-selective COX-2/COX-1 inhibitor, may be utilized as a positive control. The PGE₂ formed is typically measured by standard ELISA technology utilizing a PGE2 specific antibody, available from a number of commercial sources.

Each compound to be tested may be individually dissolved in 2 ml of dimethyl sulfoxide (DMSO) for bioassay testing to determine the COX-1 and COX-2 inhibitory effects of each particular compound. Potency is typically expressed by the IC₅₀ value expressed as g compound/ml solvent resulting in a 50% inhibition of PGE2 production. Selective inhibition of COX-2 may be determined by the IC₅₀ ratio of COX-1/COX-2.

By way of example, a primary screen may be performed in order to determine particular compounds that inhibit COX-2 at a concentration of 10 ug/ml. The compound may then be subjected to a confirmation assay to determine the extent of COX-2 inhibition at three different concentrations (e.g., 10 ug/ml, 3.3 ug/ml and 1.1 ug/ml). After this screen, compounds can then be tested for their ability to inhibit COX-1 at a concentration of 10 ug/ml. With this assay, the percentage of COX inhibition compared to control can be determined, with a higher percentage indicating a greater degree of COX inhibition. In addition, the IC₅₀ value for COX-1 and COX-2 can also be determined for the tested compound. The selectivity for each compound may then be determined by the IC₅₀ ratio of COX-1/COX-2, as set-forth above.

### Example 2

### Evaluation of the Combination Therapy in Human Subjects

This trial can be designed as a COX-2 selective inhibitor-controlled efficacy study of the combination therapy described herein in patients presenting with migraine headaches accompanied by nausea and/or vomiting. Patients are selected for the trial based on a set of eligibility criteria that can be determined for each study. For example, the patients can be selected based on the presence of migraine features that were established by IHS and described above. The exclusion criteria can include, e.g., severe coexisting systemic disease, preexisting medical conditions that may interfere with participation, and surgery that is required within 24 hours. The protocol for the study should be approved by the institutional review board of the institution where the trial is taking place and all patients or their legal representatives should sign an informed consent. The primary objective of this study is to determine the effects of the combination therapy on nausea and vomiting associated with migraine headaches.

All patients who qualify according to the inclusion and exclusion criteria and for whom informed consent is obtained are randomly allocated on a one-to-one basis to treatment with either a COX-2 selective inhibitor or combination therapy, comprising a COX-2 selective inhibitor and an anti-nausea agent. Both combination therapy and placebo can be administered orally. The therapy can be administered, e.g., starting with the onset of a migraine headache, and periodically during the migraine. It should be noted that other routes of administration and other dose schedules can readily be determined by a skilled artisan. In addition, the combination therapy and COX-2 selective inhibitor can be administered for, e.g., 5 consecutive migraine episodes that a patient experiences.

The efficacy of the combination therapy may be measured in several ways. The primary outcome measure may be, e.g., a comparison of proportion of patients in the COX-2 selective inhibitor and combination therapy groups who have noted signs of improvement in headache intensity, photophobia, phonophobia, nausea and/or vomiting. It is expected that the improvement should be more significant in patients receiving the combination treatment rather than a COX-2 selective inhibitor alone.

It should also be noted that all of the above-mentioned procedures can be modified for a particular study, depending on factors such as a drug combination used, length of the study, subjects that are selected, etc. Such modifications can be designed by a skilled artisan without undue experimentation.

1. A method for treating a migraine accompanied by nausea or vomiting, the method comprising:
(a) diagnosing a subject in need of treatment for a migraine accompanied by nausea or vomiting; and
(b) administering to the subject a cyclooxygenase-2 selective inhibitor or an isomer, a pharmaceutically acceptable salt, ester, or prodrug thereof and an anti-nausea agent or an isomer, a pharmaceutically acceptable salt, ester, or prodrug thereof.

2. The method of claim 1 wherein the cyclooxygenase-2 selective inhibitor has a selectivity ratio of COX-1 IC₅₀ to COX-2 IC₅₀ not less than about 50.

3. The method of claim 1 wherein the cyclooxygenase-2 selective inhibitor has a selectivity ratio of COX-1 IC₅₀ to COX-2 IC₅₀ not less than about 100.

4. The method of claim 1 wherein the cyclooxygenase-2 selective inhibitor is selected from the group consisting of celecoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, 2-[(2,4-dichloro-6-methylphenyl)amino]-5-ethyl-benzeneacetic acid, (3Z)-3-[(4-chlorophenyl)[4-(methylsulfonyl)phenyl]methylene]dihydro-2(3H)-furanone, and (S)-6,8-dichloro-2-(trifluoromethyl)-2H-1-benzopyran-3-carboxylic acid,
or an isomer, a pharmaceutically acceptable salt, ester, or prodrug thereof.

5. The method of claim 1 wherein the anti-nausea agent is selected from the group consisting of scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth subsalicylate, buclizine, cinnarizine, cyclizine, diphenidol, dolasetron, domperidone, dronabinol, droperidol, granisetron, haloperidol, metoclopramide, nabilone, ondansetron, thiethylperazine, trimethobenzamide, and ezlopitant,
or an isomer, a pharmaceutically acceptable salt, ester, or prodrug thereof.

6. The method of claim 4 wherein the anti-nausea agent is selected from the group consisting of scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth subsalicylate, buclizine, cinnarizine, cyclizine, diphenidol, dolasetron, domperidone, dronabinol, droperidol, granisetron, haloperidol, metoclopramide, nabilone, ondansetron, thiethylperazine, trimethobenzamide, and ezlopitant,
or an isomer, a pharmaceutically acceptable salt, ester, or prodrug thereof.

7. The method of claim 1 wherein the cyclooxygenase-2 selective inhibitor and anti-nausea agent are administered substantially simultaneously.

8. The method of claim 1 wherein the cyclooxygenase-2 selective inhibitor and anti-nausea agent are administered sequentially.

9. The method of claim 1 wherein the cyclooxygenase-2 selective inhibitor is administered to the subject in an amount of about 0.1 to about 20 mg/kg body weight per day.

10. The method of claim 1 wherein the anti-nausea agent is administered to the subject in an amount of about 5 to about 300 milligrams per day.

11. A method for treating a migraine accompanied by nausea or vomiting, the method comprising:
(a) diagnosing a subject in need of treatment for a migraine accompanied by nausea or vomiting; and
(b) administering to the subject an anti-nausea agent or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a chromene compound, the chromene compound comprising a benzothiopyran, a dihydroquinoline or a dihydronaphthalene.

12. The method of claim 11 wherein the cyclooxygenase-2 selective inhibitor has a selectivity ratio of COX-1 IC₅₀ to COX-2 IC₅₀ not less than about 50.

13. The method of claim 11 wherein the cyclooxygenase-2 selective inhibitor has a selectivity ratio of COX-1 IC₅₀ to COX-2 IC₅₀ not less than about 100.

14. The method of claim 11 wherein the cyclooxygenase-2 selective inhibitor is a compound having the formula: 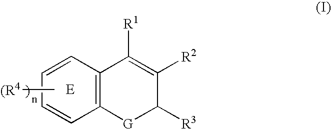
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical,
or an isomer, a pharmaceutically acceptable salt, ester, or prodrug thereof.

15. The method of claim 11 wherein the cyclooxygenase-2 selective inhibitor is (S)-6,8-dichloro-2-(trifluoromethyl)-2H-1-benzopyran-3-carboxylic acid.

16. The method of claim 11 wherein the anti-nausea agent is selected from the group consisting of scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth subsalicylate, buclizine, cinnarizine, cyclizine, diphenidol, dolasetron, domperidone, dronabinol, droperidol, granisetron, haloperidol, metoclopramide, nabilone, ondansetron, thiethylperazine, trimethobenzamide, and eziopitant,
or an isomer, a pharmaceutically acceptable salt, ester, or prodrug thereof.

17. A method for treating a migraine accompanied by nausea or vomiting, the method comprising:
(a) diagnosing a subject in need of treatment for a migraine accompanied by nausea or vomiting; and
(b) administering to the subject an anti-nausea agent or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a tricyclic compound, the tricyclic compound containing a benzenesulfonamide or methylsulfonylbenzene moiety.

18. The method of claim 17 wherein the cyclooxygenase-2 selective inhibitor has a selectivity ratio of COX-1 IC₅₀ to COX-2 IC₅₀ not less than about 50.

19. The method of claim 17 wherein the cyclooxygenase-2 selective inhibitor has a selectivity ratio of COX-1 IC₅₀ to COX-2 IC₅₀ not less than about 100.

20. The method of claim 17 wherein the cyclooxygenase-2 selective inhibitor is a compound of the formula: 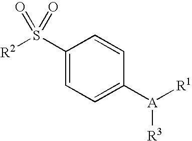
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl,
or an isomer, a pharmaceutically acceptable salt, ester, or prodrug thereof.

21. The method of claim 17 wherein the cyclooxygenase-2 selective inhibitor is selected from the group consisting of celecoxib, valdecoxib, parecoxib, deracoxib, rofecoxib, etoricoxib, and 2-(3,4-difluorophenyl)-4-(3-hydroxy-3-methylbutoxy)-5-[4-(methylsulfonyl)phenyl]-3(2H)-pyridazinone,
or an isomer, a pharmaceutically acceptable salt, ester, or prodrug thereof.

22. The method of claim 17 wherein the anti-nausea agent is selected from the group consisting of scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth subsalicylate, buclizine, cinnarizine, cyclizine, diphenidol, dolasetron, domperidone, dronabinol, droperidol, granisetron, haloperidol, metoclopramide, nabilone, ondansetron, thiethylperazine, trimethobenzamide, and ezlopitant,
or is an isomer, a pharmaceutically acceptable salt, ester, or prodrug thereof.

23. A method for treating a migraine accompanied by nausea or vomiting, the method comprising:
(a) diagnosing a subject in need of treatment for a migraine accompanied by nausea or vomiting; and
(b) administering to the subject an anti-nausea agent or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a phenyl acetic acid compound.

24. The method of claim 23 wherein the cyclooxygenase-2 selective inhibitor has a selectivity ratio of COX-1 IC₅₀ to COX-2 IC₅₀ not less than about 50.

25. The method of claim 23 wherein the cyclooxygenase-2 selective inhibitor has a selectivity ratio of COX-1 IC₅₀ to COX-2 IC₅₀ not less than about 100.

26. The method of claim 23 wherein the cyclooxygenase-2 selective inhibitor is a compound having the formula: 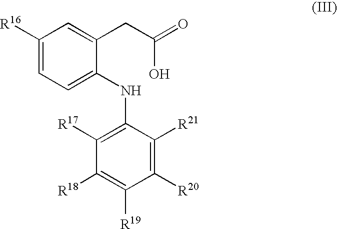
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl; provided, however, that each of R¹⁷, R¹⁸, R²⁰ and R²¹ is not fluoro when R¹⁶ is ethyl and R¹⁹ is H.

27. The method of claim 26 wherein:
R¹⁶ is ethyl;
R¹⁷ and R¹⁹ are chloro;
R¹⁸ and R²⁰ are hydrogen; and
R²¹ is methyl.

28. The method of claim 23 wherein the anti-nausea agent is selected from the group consisting of scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth subsalicylate, buclizine, cinnarizine, cyclizine, diphenidol, dolasetron, domperidone, dronabinol, droperidol, granisetron, haloperidol, metoclopramide, nabilone, ondansetron, thiethylperazine, trimethobenzamide, and eziopitant,
or is an isomer, a pharmaceutically acceptable salt, ester, or prodrug thereof.

29. A method for treating a migraine accompanied by nausea or vomiting, the method comprising:
(a) diagnosing a subject in need of treatment for a migraine accompanied by nausea or vomiting; and
(b) administering to the subject a cyclooxygenase-2 selective inhibitor selected from the group consisting of celecoxib, deracoxib, valdecoxib, rofecoxib, lumiracoxib, etoricoxib, parecoxib, 2-(3,4-difluorophenyl)-4-(3-hydroxy-3-methylbutoxy)-5-[4-(methylsulfonyl)phenyl]-3(2H)-pyridazinone, and (S)-6,8-dichloro-2-(trifluoromethyl)-2H-1-benzopyran-3-carboxylic acid, or an isomer, a pharmaceutically acceptable salt, ester, or prodrug thereof; and
an anti-nausea agent selected from the group consisting of scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth subsalicylate, buclizine, cinnarizine, cyclizine, diphenidol, dolasetron, domperidone, dronabinol, droperidol, granisetron, haloperidol, metoclopramide, nabilone, ondansetron, thiethylperazine, trimethobenzamide, and eziopitant,
or an isomer, a pharmaceutically acceptable salt, ester, or prodrug thereof.

30. The method of claim 29 wherein the cyclooxygenase-2 selective inhibitor and the anti-nausea agent are combined and administered in the same dose.

31. The method of claim 29 wherein the cyclooxygenase-2 selective inhibitor and the anti-nausea agent are administered in separate doses.
